(19) United States
(12) Patent Application Publication
(10) Pub. No.: US 2009/0304817 A1
(43) Pub. Date: Dec. 10, 2009

(54) Glycosylation Variants of Ricin-Like Proteins

(76) Inventors: Thor Borgford, New Westminster (CA); Curtis Braun, Surrey (CA); Admir Purac, Burnaby (CA); Dominik Stoll, Vancouver (CA)

Correspondence Address: BERESKIN AND PARR LLP/S.E.N.C.R.L., s.r.l., 40 KING STREET WEST, BOX 401, TORONTO, ON, M5H 3Y2, CA

(21) Appl. No.: 10/593,958

(22) PCT Filed: Mar. 24, 2005
(86) PCT No.: PCT/CA2005/000436

§ 371 Date: Mar. 20, 2009

Related U.S. Application Data
(60) Provisional application No. 60/555,678, filed on Mar. 24, 2004.

(51) Int. Cl. A61K 38/16; C07K 14/195; C12N 15/31; C12N 5/00; C12P 21/02; A61K 33/24; A61P 35/00

(52) U.S. Cl. 424/649; 530/402; 536/23.1; 435/377; 435/70.1; 514/12

(57) ABSTRACT

The present invention provides glycosylation variants of recombinant proteins and nucleic acids that encode such recombinant proteins, which are useful as therapeutics against cancer, and viral, parasitic and fungal infections. The proteins and nucleic acids have A and B chains of ricin-like toxin linked by a linker sequence that is specifically cleaved and activated by proteases specific to disease-associated pathogens or cells. The invention also relates to methods of inhibiting or destroying cells affected by a disease, methods of treating a mammal with a disease, and pharmaceutical compositions using the recombinant proteins and nucleic acids of the invention.

### FIELD OF THE INVENTION

The invention relates to glycosylation variants of recombinant proteins and nucleic acids that encode such recombinant proteins, which are useful as therapeutics against cancer, and viral, parasitic and fungal infections. The proteins and nucleic acids have A and B chains of ricin-like toxin linked by a linker sequence that is specifically cleaved and activated by proteases specific to disease-associated pathogens or cells.

### BACKGROUND OF THE INVENTION

Bacteria and plants are known to produce cytotoxic proteins which may consist of one, two or several polypeptides or subunits. Those proteins having a single subunit may be loosely classified as Type I proteins. Many of the cytotoxins which have evolved two subunit structures are referred to as type II proteins (Saelinger, C. B. in Trafficking of Bacterial Toxins (eds. Saelinger, C. B.) 1-13 (CRC Press Inc., Boca Raton, Fla., 1990). One subunit, the A chain, possesses the toxic activity whereas the second subunit, the B chain, binds cell surfaces and mediates entry of the toxin into a target cell. A subset of these toxins kill target cells by inhibiting protein biosynthesis. For example, bacterial toxins such as diphtheria toxin or Pseudomonas exotoxin inhibit protein synthesis by inactivating elongation factor 2. Plant toxins such as ricin, abrin, and bacterial toxin Shiga toxin, inhibit protein synthesis by directly inactivating the ribosomes (Olsnes, S. & Phil, A. in Molecular action of toxins and viruses (eds. Cohen, P. & vanHeyningen, S.) 51-105 Elsevier Biomedical Press, Amsterdam, 1982).

Ricin, derived from the seeds of Ricinus communis (castor oil plant), may be the most potent of the plant toxins. It is estimated that a single ricin A chain is able to inactivate ribosomes at a rate of 1500 ribosomes/minute. Consequently, a single molecule of ricin is enough to kill a cell (Olsnes, S. & Phil, A. in Molecular action of toxins and viruses (eds. Cohen, P. & vanHeyningen, S.) (Elsevier Biomedical Press, Amsterdam, 1982). The ricin toxin is a glycosylated heterodimer consisting of A and B chains with molecular masses of 30,625 Da and 31,431 Da linked by a disulphide bond. The A chain of ricin has an N-glycosidase activity and catalyzes the excision of a specific adenine residue from the 28S rRNA of eukaryotic ribosomes (Endo, Y. & Tsurugi, K. J., Biol. Chem. 262:8128 (1987)). The B chain of ricin, although not toxic in itself, promotes the toxicity of the A chain by binding to galactose residues on the surface of eukaryotic cells and stimulating receptor-mediated endocytosis of the toxin molecule (Simmons et al., Biol. Chem. 261:7912 (1986)). Once the toxin molecule consisting of the A and B chains is internalized into the cell via clathrin-dependent or independent mechanisms, the greater reduction potential within the cell induces a release of the active A chain, eliciting its inhibitory effect on protein synthesis and its cytotoxicity (Emmanuel, F. et al., Anal. Biochem. 173: 134-141 (1988); Blum, J. S. et al., J. Biol. Chem. 266: 22091-22095 (1991); Fiani, M. L. et al., Arch. Biochem. Biophys. 307: 225-230 (1993)). Empirical evidence suggests that activated toxin (e.g. ricin, shiga toxin and others) in the endosomes is transcytosed through the trans-Golgi network to the endoplasmic reticulum by retrograde transport before the A chain is translocated into the cytoplasm to elicit its action (Sandvig, K. & van Deurs, B., FEBS Lett. 346: 99-102 (1994).

Protein toxins are initially produced in an inactive, precursor form. Ricin is initially produced as a single polypeptide (preproricin) with a 35 amino acid N-terminal presequence and 12 amino acid linker between the A and B chains. The pre-sequence is removed during translocation of the ricin precursor into the endoplasmic reticulum (Lord, J. M., Eur. J. Biochem. 146:403-409 (1985) and Lord, J. M., Eur. J. Biochem. 146:411-416 (1985)). The proricin is then translocated into specialized organelles called protein bodies where a plant protease cleaves the protein at a linker region between the A and B chains (Lord, J. M. et al., FASAB Journal 8:201-208 (1994)). The two chains, however, remain covalently attached by an interchain disulfide bond (cysteine 259 in the A chain to cysteine 4 in the B chain) and mature disulfide linked ricin is stored in protein bodies inside the plant cells. The A chain is inactive in proricin (O'Hare, M. et al., FEBS Lett. 273:200-204 (1990)) and it is inactive in the disulfide-linked mature ricin (Richardson, P. T. et al., FEBS Lett. 255:15-20 (1989)). The ribosomes of the castor bean plant are themselves susceptible to inactivation by ricin A chain; however, as there is no cell surface galactose to permit B chain recognition the A chain cannot re-enter the cell. The exact mechanism of A chain release and activation in target cell cytoplasm is not known (Lord, J. M. et al., FASAB Journal 8:201-208 (1994)). However, it is known that for activation to take place the disulfide bond between the A and B chains must be reduced and, hence, the linkage between subunits broken.

Diphtheria toxin is produced by Corynebacterium diphtheriae as a 535 amino acid polypeptide with a molecular weight of approximately 58 kD (Greenfield, L. et al., Proc. Natl. Acad. Sci. USA 80:6853-6857 (1983); Pastan, I. et al., Annu. Rev. Biochem. 61:331-354 (1992); Collier, R. J. & Kandel, J., J. Biol. Chem. 246:1496-1503 (1971)). It is secreted as a single chain polypeptide consisting of 2 functional domains. Similar to proricin, the N-terminal domain (A-chain) contains the cytotoxic moiety whereas the C-terminal domain (B-chain) is responsible for binding to the cells and facilitates toxin endocytosis. Conversely, the mechanism of cytotoxicity for diphtheria toxin is based on ADP-ribosylation of EF-2 thereby blocking protein synthesis and producing cell death. The 2 functional domains in diphtheria toxin are linked by an arginine-rich peptide sequence as well as a disulphide bond. Once the diphtheria toxin is internalized into the cell, the arginine-rich peptide linker is cleaved by trypsin-like enzymes and the disulphide bond (Cys 186-201) is reduced. The cytotoxic domain is subsequently translocated into the cytosol substantially as described above for ricin and elicits ribosomal inhibition and cytotoxicity.

Pseudomonas exotoxin is also a 66 kD single-chain toxin protein secreted by Pseudomonas aeruginosa with a similar mechanism of cytotoxicity to that of diphtheria toxin (Pastan, I. et al., Annu. Rev. Biochem. 61:331-354 (1992); Ogata, M. et al., J. Biol. Chem. 267:25396-25401 (1992); Vagil, M. L. et al., Infect. Immunol. 16:353-361 (1977)). Pseudomonas exotoxin consists of 3 conjoint functional domains. The first domain Ia (amino acids 1-252) is responsible for cell binding and toxin endocytosis, a second domain II (amino acids 253-364) is responsible for toxin translocation from the endocytic vesicle to the cytosol, and a third domain III (amino acids 400-613) is responsible for protein synthesis inhibition and cytotoxicity. After Pseudomonas exotoxin enters the cell, the liberation of the cytotoxic domain is effected by both proteolytic cleavage of a polypeptide sequence in the second domain (near Arg 279) and the reduction of the disulphide bond (Cys 265-287) in the endocytic vesicles. In essence, the overall pathway to cytotoxicity is analogous to diphtheria toxin with the exception that the toxin translocation domain in Pseudomonas exotoxin is structurally distinct.

Class 2 ribosomal inhibitory proteins (RIP-2) constitute other toxins possessing distinct functional domains for cytotoxicity and cell binding/toxin translocation which include abrin, modeccin, volkensin, (Sandvig, K. et al., Biochem. Soc. Trans. 21:707-711 (1993)) and mistletoe lectin (viscumin) (Olsnes, S. & Phil, A. in Molecular action of toxins and viruses (eds. Cohen, P. & vanHeyningen, S.) 51-105 Elsevier Biomedical Press, Amsterdam, 1982; Fodstad, et al. Canc. Res. 44: 862 (1984)). Some toxins such as Shiga toxin and cholera toxin also have multiple polypeptide chains responsible for receptor binding and endocytosis.

The ricin gene has been cloned and sequenced, and the X-ray crystal structures of the A and B chains have been described (Rutenber, E. et al. Proteins 10:240-250 (1991); Weston et al., Mol. Bio. 244:410-422, 1994; Lamb and Lord, Eur. J. Biochem. 14:265 (1985); Hailing, K. et al. Nucleic Acids Res. 13:8019 (1985)). Similarly, the genes for diphtheria toxin and Pseudomonas exotoxin have been cloned and sequenced, and the 3-dimensional structures of the toxin proteins have been elucidated and described (Columblatti, M. et al., J. Biol. Chem. 261:3030-3035 (1986); Allured, V. S. et al., Proc. Natl. Acad. Sci. USA 83:1320-1324 (1986); Gray, G. L. et al., Proc. Natl. Acad. Sci. USA 81:2645-2649 (1984); Greenfield, L. et al., Proc. Natl. Acad. Sci. USA 80:6853-6857 (1983); Collier, R. J. et al., J. Biol. Chem. 257:5283-5285 (1982)).

Ricin-like toxins have been shown to be useful for treating viral infections, cancer, and parasitic and fungal inventions (U.S. Pat. Nos. 6,333,303; 6,531,125; and 6,593,132 are incorporated herein by reference). Bacterial toxins such as Pseudomonas exotoxin-A and subunit A of diphtheria toxin; dual chain ribosomal inhibitory plant toxins such as ricin, and single chain ribosomal inhibitory proteins such as trichosanthin and pokeweed antiviral protein have been used for the elimination of HIV infected cells (Olson et al., AIDS Res. and Human Retroviruses 7:1025-1030 (1991)). The high toxicity of these toxins for mammalian cells, combined with a lack of specificity of action poses a major problem to the development of pharmaceuticals incorporating the toxins, such as immunotoxins.

Due to their extreme toxicity there has been much interest in making ricin-based immunotoxins as therapeutic agents for specifically destroying or inhibiting infected or tumourous cells or tissues (Vitetta et al., Science 238:1098-1104 (1987)). An immunotoxin is a conjugate of a specific cell binding component, such as a monoclonal antibody or growth factor and the toxin in which the two protein components are covalently linked. Generally, the components are chemically coupled. However, the linkage may also be a peptide or disulfide bond. The antibody directs the toxin to cell types presenting a specific antigen thereby providing a specificity of action not possible with the natural toxin. Immunotoxins have been made both with the entire ricin molecule (i.e. both chains) and with the ricin A chain alone (Spooner et al., Mol. Immunol. 31:117-125, (1994)).

Immunotoxins made with the ricin dimer (IT-RS) are more potent toxins than those made with only the A chain (IT-As). The increased toxicity of IT-RS is thought to be attributed to the dual role of the B chains in binding to the cell surface and in translocating the A chain to the cytosolic compartment of the target cell (Vitetta et al., Science 238:1098 1104 (1987); Vitetta & Thorpe, Seminars in Cell Biology 2:47-58 (1991)). However, the presence of the B chain in these conjugates also promotes the entry of the immunotoxin into nontarget cells. Even small amounts of B chain may override the specificity of the cell-binding component as the B chain will bind nonspecifically to galactose associated with N-linked carbohydrates, which is present on most cells. IT-As are more specific and safer to use than IT-RS. However, in the absence of the B chain the A chain has greatly reduced toxicity. Due to the reduced potency of IT-As as compared to IT-RS, large doses of IT-As must be administered to patients. The large doses frequently cause immune responses and production of neutralizing antibodies in patients (Vitetta et al., Science 238:1098-1104 (1987)). IT-As and IT-RS both suffer from reduced toxicity as the A chain is not released from the conjugate into the target cell cytoplasm.

A number of immunotoxins have been designed to recognize antigens on the surfaces of tumour cells and cells of the immune system (Pastan et al., Annals New York Academy of Sciences 758:345-353 (1995)). A major problem with the use of such immunotoxins is that the antibody component is its only targeting mechanism and the target antigen is often found on non-target cells (Vitetta et al., Immunology Today 14:252-259 (1993)). Also, the preparation of a suitable specific cell binding component may be problematic. For example, antigens specific for the target cell may not be available and many potential target cells and infective organisms can alter their antigenic make up rapidly to avoid immune recognition. In view of the extreme toxicity of proteins such as ricin, the lack of specificity of the immunotoxins may severely limit their usefulness as therapeutics for the treatment of cancer and infectious diseases.

The insertion of intramolecular protease cleavage sites between the cytotoxic and cell-binding components of a toxin can mimic the way that the natural toxin is activated. European patent application no. 466,222 describes the use of maize-derived pro-proteins which can be converted into active form by cleavage with extracellular blood enzymes such as factor Xa, thrombin or collagenase. Garred, O. et al. (J. Biol. Chem. 270:10817-10821 (1995)) documented the use of a ubiquitous calcium dependent serine protease, furin, to activate shiga toxin by cleavage of the trypsin-sensitive linkage between the cytotoxic A-chain and the pentamer of cell-binding B-units. Westby et al. (Bioconjugate Chem. 3:375-381 (1992)) documented fusion proteins which have a specific cell binding component and proricin with a protease sensitive cleavage site specific for factor Xa within the linker sequence. O'Hare et al. (FEBS Lett. 273:200-204 (1990)) also described a recombinant fusion protein of RTA and staphylococcal protein A joined by a trypsin-sensitive cleavage site. In view of the ubiquitous nature of the extracellular proteases utilized in these approaches, such artificial activation of the toxin precursor or immunotoxin does not confer a mechanism for intracellular toxin activation and the problems of target specificity and adverse immunological reactions to the cell-binding component of the immunotoxin remain.

In a variation of the approach of insertion of intramolecular protease cleavage sites on proteins which combine a binding chain and a toxic chain, Leppla, S. H. et al. (Bacterial Protein Toxins zbl.bakt.suppl. 24:431-442 (1994)) suggest the replacement of the native cleavage site of the protective antigen (PA) produced by Bacillus anthracis with a cleavage site that is recognized by cells that contain a particular protease. PA, recognizes, binds, and thereby assists in the internalization of lethal factor (LF) and edema toxin (ET). also produced by Bacillus anthracis. However, this approach is wholly dependent on the availability of LF, or ET and PA all being localized to cells wherein the modified PA can be activated by the specific protease. It does not confer a mechanism for intracellular toxin activation and presents a problem of ensuring sufficient quantities of toxin for internalization in target cells.

The in vitro activation of a Staphylococcus-derived pore forming toxin, α-hemolysin by extracellular tumour-associated proteases has been documented (Panchel, R. G. et al., Nature Biotechnology 14:852-857 (1996)). Artificial activation of α-hemolysin in vitro by said proteases was reported but the actual activity and utility of α-hemolysin in the destruction of target cells were not demonstrated.

α-Hemolysin does not inhibit protein synthesis but is a heptameric transmembrane pore which acts as a channel to allow leakage of molecules up to 3 kD thereby disrupting the ionic balances of the living cell. The α-hemolysin activation domain is likely located on the outside of the target cell (for activation by extracellular proteases). The triggering mechanism in the disclosed hemolysin precursor does not involve the intracellular proteolytic cleavage of 2 functionally distinct domains. Also, the proteases used for the α-hemolysin activation are ubiquitously secreted extracellular proteases and toxin activation would not be confined to activation in the vicinity of diseased cells. Such widespread activation of the toxin does not confer target specificity and limits the usefulness of said α-hemolysin toxin as therapeutics due to systemic toxicity.

A variety of proteases specifically associated with malignancy, viral infections and parasitic infections have been identified and described. For example, cathepsin is a family of serine, cysteine or aspartic endopeptidases and exopeptidases which has been implicated to play a primary role in cancer metastasis (Schwartz, M. K., Clin. Chim. Acta 237:67-78 (1995); Spiess, E. et al., J. Histochem. Cytochem. 42:917-929 (1994); Scarborough, P. E. et al., Protein Sci. 2:264-276 (1993); Sloane, B. F. et al., Proc. Natl. Acad. Sci. USA 83:2483-2487 (1986); Mikkelsen, T. et al., J. Neurosurge 83:285-290 (1995)). Matrix metalloproteinases (MMPs or matrixins) are zinc-dependent proteinases consisting of collagenases, matrilysin, stromelysins, gelatinases and macrophage elastase (Krane, S. M., Ann. N.Y. Acad. Sci. 732:1-10 (1994); Woessner, J. F., Ann. N.Y. Acad. Sci. 732:11-21 (1994); Carvalho, K. et al., Biochem. Biophys. Res. Comm. 191:172-179 (1993); Nakano, A. et al. J. of Neurosurge, 83:298-307 (1995); Peng, K-W, et al. Human Gene Therapy, 8:729-738 (1997); More, D. H. et al. Gynaecologic Oncology, 65:78-82 (1997)). These proteases are involved in pathological matrix remodeling. Under normal physiological conditions, regulation of matrixin activity is effected at the level of gene expression. Enzymatic activity is also controlled stringently by tissue inhibitors of metalloproteinases (TIMPs) (Murphy, G. et al., Ann. N.Y. Acad. Sci. 732:31 41 (1994)). The expression of MMP genes is reported to be activated in inflammatory disorders (e.g. rheumatoid arthritis) and malignancy.

In malaria, parasitic serine and aspartic proteases are involved in host erythrocyte invasion by the Plasmodium parasite and in hemoglobin catabolism by intraerythrocytic malaria (O'Dea, K. P. et al., Mol. Biochem. Parasitol. 72:111-119 (1995); Blackman, M. J. et al., Mol. Biochem. Parasitol. 62:103-114 (1993); Cooper, J. A. et al., Mol. Biochem. Parasitol. 56:151 160 (1992); Goldberg, D. E. et al., J. Exp. Med. 173:961-969 (1991)). Schistosoma mansoni is also a pathogenic parasite which causes schistosomiasis or bilharzia. Elastinolytic proteinases have been associated specifically with the virulence of this particular parasite (McKerrow, J. H. et al., J. Biol. Chem. 260:3703-3707 (1985)).

Welch, A. R. et al. (Proc. Natl. Acad. Sci. USA 88:10797-10800 (1991)) has described a series of viral proteases which are specifically associated with human cytomegalovirus, human herpesviruses, Epstein Barr virus, varicella zoster virus-I. and infectious laryngotracheitis virus. These proteases possess similar substrate specificity and play an integral role in viral scaffold protein restructuring in capsid assembly and virus maturation. Other viral proteases serving similar functions have also been documented for human T-cell leukemia virus (Blaha, I. et al., FEBS Lett. 309:389-393 (1992); Pettit, S. C. et al., J. Biol. Chem. 266:14539-14547 (1991)), hepatitis viruses (Hirowatari, Y. et al., Anal. Biochem. 225:113-120 (1995); Hirowatari, Y. et al., Arch. Virol. 133:349-356 (1993); Jewell, D. A. et al., Biochemistry 31:7862-7869 (1992)), poliomyelitis virus (Weidner, J. R. et al., Arch. Biochem. Biophys. 286:402-408 (1991)), and human rhinovirus (Long, A. C. et al., FEBS Lett. 258:75-78 (1989)).

Candida yeasts are dimorphic fungi which are responsible for a majority of opportunistic infections in AIDS patients (Holmberg, K. and Myer, R., Scand. J. Infect. Dis. 18:179-192 (1986)). Aspartic proteinases have been associated specifically with numerous virulent strains of Candida including Candida albican, Candida tropicalis, and Candida parapsilosis (Abad Zapatero, C. et al., Protein Sci. 5:640-652 (1996); Cutfield, S. M. et al., Biochemistry 35:398-410 (1995); Ruchel, R. et al, Zentralbl. Bakteriol. Mikrobiol Hyg. I Abt Orig. A. 255:537-548 (1983); Remold, H. et al., Biochim. Biophys. Acta 167:399-406 (1968)), and the levels of these enzymes have been correlated with the lethality of the strain (Schreiber, B, et al., Diagn. Microbiol. Infect. Dis. 3:1-5 (1985)).

### SUMMARY OF THE INVENTION

Ricin is a glycoprotein possessing N-linked carbohydrate. According to the amino acid sequence of ricin there are four potential sites of carbohydrate attachment (sequons). There are two sites in the A-chain and two sites in the B-chain. To some extent glycosylation occurs at all four sites in the natural protein. The importance of glycosylation to the stability and activity of the molecule is not entirely clear. The present inventors have prepared and examined glycosylation variants of ricin-like proteins. The inventors have determined that recombinant proteins containing one glycosylation site are active and stable while proteins with no glycosylation sites are much less active and much less stable.

In one aspect the present invention provides a recombinant protein comprising (a) an A chain of a ricin-like toxin, (b) a B chain of a ricin-like toxin and (c) a heterologous linker amino acid sequence linking the A and B chains, the linker sequence containing a cleavage recognition site for a disease-specific protease, wherein the A chain or the B chain has at least one glycosylation site. In a preferred embodiment of the invention the B chain has at least one glycosylation site. In another preferred embodiment of the invention, the B chain is glycosylated at B1.

In another embodiment of the invention the recombinant protein has a linker amino acid sequence of not greater than 10 amino acids, preferably not greater than 9 amino acids or, most preferably 8 amino acids in length.

A further embodiment of the invention provides the recombinant protein with a ricin secretion signal sequence.

In a preferred embodiment of the invention the recombinant protein has the amino acid sequence shown in FIG. 1, 2 or 3 (SEQ ID Nos. 1-3).

Another aspect of the invention provides a purified and isolated nucleic acid molecule comprising (a) a nucleotide sequence encoding an A chain of a ricin-like toxin, (b) a nucleotide sequence encoding a B chain of a ricin-like toxin and (c) a nucleotide sequence encoding a heterologous linker amino acid sequence linking the A and B chain, the heterologous linker sequence containing a cleavable recognition site for a disease-specific protease, wherein the nucleotide sequence encoding the A chain or the nucleotide sequence encoding the B chain encodes at least one amino acid having a glycosylation site. In a preferred embodiment of the invention, the nucleotide sequence of the B chain encodes at least one amino acid having a glycosylation site. In another preferred embodiment the nucleotide sequence of the B chain encodes an amino acid at B1 having a glycosylation site.

In another embodiment of the invention the nucleic acid molecule encodes a linker amino acid sequence of not greater than 10 amino acids, preferably not greater than 9 amino acids, or most preferably 8 amino acids in length.

A further embodiment provides a nucleic acid molecule of the invention that encodes a ricin secretion signal sequence.

In a preferred embodiment of the invention the nucleic acid molecule has the sequence as shown in FIG. 4, 5 or 6 (SEQ ID Nos. 4-6).

The heterologous linker, which links the A chain and the B chain, may be cleaved specifically by a protease localized in cells or tissues affected by a specific disease to liberate toxic A chain thereby selectively inhibiting or destroying the diseased cells or tissues. The term diseased cells as used herein, includes cells cancer cells, or cells infected by fungi, parasites or viruses, including retroviruses.

Toxin targeting using the recombinant toxic proteins of the invention takes advantage of the fact that many DNA viruses exploit host cellular transport mechanisms to escape immunological destruction. This is achieved by enhancing the retrograde translocation of host major histocompatibility complex (MHC) type 1 molecules from the endoplasmic reticulum into the cytoplasm (Bonifacino, J. S., Nature 384: 405-406 (1996); Wiertz, E. J. et al., Nature 384: 432-438 (1996)). The facilitation of retrograde transport in diseased cells by the virus can enhance the transcytosis and cytotoxicity of a recombinant toxic protein of the present invention thereby further reducing non-specific cytotoxicity and improving the overall safety of the product.

The recombinant toxic proteins of the present invention may be used to treat diseases including various forms of cancer such as T- and B-cell lymphoproliferative diseases, ovarian cancer, pancreatic cancer, head and neck cancer, squamous cell carcinoma, gastrointestinal cancer, breast cancer, prostate cancer, lung cancer, liver cancer, malaria, and diverse viral disease states associated with infection such as human cytomegalovirus, hepatitis virus, herpes virus, human rhinovirus, infectious laryngotracheitis virus, poliomyelitis virus, or varicella zoster virus.

One aspect of the invention provides a method of inhibiting or destroying cells affected by a disease, which cells are associated with a disease specific protease, including cancer or infection with a virus, fungus, or a parasite each of which has a specific protease, comprising the steps of preparing a recombinant protein of the invention having a heterologous linker sequence which contains a cleavage recognition site for the disease-specific protease and administering the recombinant protein to the cells. In an embodiment, the cancer is T-cell or B-cell lymphoproliferative disease, ovarian cancer, pancreatic cancer, head and neck cancer, squamous cell carcinoma, gastrointestinal cancer, breast cancer, prostate cancer, lung cancer, and liver cancer. In another embodiment, the virus is human cytomegalovirus, Epstein-Barr virus, hepatitis virus, herpes virus, human rhinovirus, human T-cell leukemia virus, infectious laryngotracheitis virus, poliomyelitis virus, or varicella zoster virus. In another embodiment, the parasite is Plasmodium falciparum.

The present invention also relates to a method of treating a mammal with disease wherein cells affected by the disease are associated with a disease specific protease, including cancer or infection with a virus, a fungus, or a parasite each of which has a specific protease, by administering an effective amount of one or more recombinant proteins of the invention to said mammal.

Still further, a process is provided for preparing a pharmaceutical for treating a mammal with disease wherein cells affected by the disease are associated with a disease specific protease, including cancer or infection with a virus, a fungus, or a parasite each of which has a specific protease comprising the steps of preparing a purified and isolated nucleic acid molecule of the invention; introducing the nucleic acid into a host cell; expressing the nucleic acid in the host cell to obtain the recombinant protein of the invention; and suspending the protein in a pharmaceutically acceptable carrier, diluent or excipient.

In an embodiment, a process is provided for preparing a pharmaceutical for treating a mammal with disease wherein cells affected by the disease are associated with a disease specific protease, including cancer or infection with a virus, a fungus, or a parasite each of which has a specific protease comprising the steps of identifying a cleavage recognition site for the protease; preparing a recombinant protein of the invention comprising an A chain of a ricin-like toxin, a B chain of a ricin-like toxin and a heterologous linker amino acid sequence, linking the A and B chains wherein the linker sequence contains the cleavage recognition site for the protease and suspending the protein in a pharmaceutically acceptable carrier, diluent or excipient.

In a further aspect, the invention provides a pharmaceutical composition for treating a mammal with disease wherein cells affected by the disease are associated with a disease specific protease, including cancer or infection with a virus, a fungus, or a parasite comprising the recombinant protein of the invention and a pharmaceutically acceptable carrier, diluent or excipient.

Another aspect of the invention is combination therapy. For example, combination therapy can be used in methods of inhibiting or destroying cancer cells or methods of treating cancer. In one embodiment, at least one conventional anticancer therapy can be included in the process for preparing a pharmaceutical composition of the invention for treating a mammal with cancer. The invention also contemplates a pharmaceutical composition of the invention for treating cancer which includes at least one additional anticancer therapy. Additional anticancer therapies include doxorubicin, cisplatin, cyclophosphamide, etoposide, paclitaxel, taxotere, carboplatin, oxaliplatin, 5-fluorouracil, irinotecan and topotecan.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will now be described in relation to the drawings in which:

FIG. 1 is the TST10088 protein sequence (SEQ ID No. 1).

FIG. 2 is the TST10092 protein sequence (SEQ ID No. 2).

FIG. 3 is the TST10147 protein sequence (SEQ ID No. 3).

FIG. 4 is the TST10088 DNA insert sequence (SEQ ID No. 4).

FIG. 5 is the TST10092 DNA insert sequence (SEQ ID No. 5).

FIG. 6 is the TST10147 DNA insert sequence (SEQ ID No. 6).

FIG. 7 shows the combinatorial mutagenesis of glycosylation, natural gene sequence.

FIG. 8 shows the glycosylation pattern from glycosylation variants.

FIG. 9 shows the efficacy of glycoform 0 against P388.

FIG. 10 shows the efficacy of glycoform 1 against P388.

FIG. 11 shows the efficacy of glycoform 2 against P388.

FIG. 12 shows weight loss data after treatment with different glycoforms.

FIG. 13 shows the glycosylation pattern from glycosylation iterative refinement variants.

FIG. 14 shows a comparison of TST10088 and Ricin cytotoxicities against COS-1 cells.

FIG. 15 shows the efficacy of TST10007 in combination with Cisplatin against P388.

FIGS. 16 A & B show the combination efficacy of TST10007/Dox in P388 model.

FIGS. 17 A & B show the combination efficacy of TST10088/Dox in P388 model.

FIGS. 18 A & B show the combination efficacy of TST10088/Cis in P388 tumour model.

FIG. 19 shows the combination efficacy of TST10088/CPA in P388 tumour model.

FIG. 20 shows the combination efficacy of TST10088/CPA in P388 tumour model.

FIG. 21 shows the kinetics of TST10088 clearance from mouse serum.

FIG. 22 shows the distribution of ¹²⁵I labeled TST10088 (Day 4 injection).

FIG. 23 shows the distribution of ¹²⁵I labeled TST10088 at 60 minutes post injection (Day 4 injection).

FIG. 24 shows the level of TST10088 in tumours with and without Doxorubicin.

FIG. 25 shows the presence of serum antibodies after treatment with TST10007 and Doxorubicin.

### DETAILED DESCRIPTION OF THE INVENTION

### (A) Recombinant Proteins of the Invention

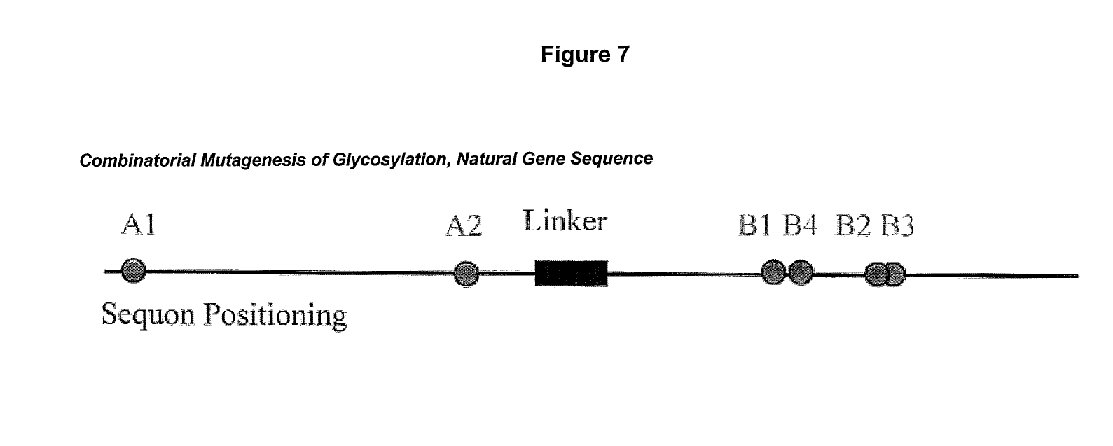

The invention provides glycosylation variants of recombinant proteins, which are useful as therapeutics against cancer, and viral, parasitic and fungal infections. Natural Ricin is a glycoprotein possessing N-linked carbohydrate. N-linked glycosylation generally occurs at a conserved sequon. However, not all sequons are actually glycosylated. According to the amino acid sequence of ricin there are four sequons: two sites in the A-chain (A1 and A2) and two sites in the B-chain (B1 and B2) (See FIG. 7). In the proricin construct with an 8 amino acid linker, the A1 glycosylation site is at amino acid position 14, the A2 glycosylation site is at amino acid position 240, the B1 glycosylation site is at amino acid position 363 and the B2 glycosylation site is at amino acid position 403.

The inventors examined 32 glycosylation variants where sequons were modified or removed. The activity and toxicity of the glycosylation variants were studied. The inventors found that a minimum of one carbohydrate chain is essential to the function of the recombinant protein. While not wishing to be bound by a particular theory, the inventors hypothesize that the attached carbohydrate determines the route of protein uptake into a target cell. Therefore, a protein devoid of carbohydrate becomes misdirected in such a way that its activity is diminished or lost. The inventors also established that, at the other extreme, extensive glycosylation increases toxicity of the molecule relative to less glycosylated species.

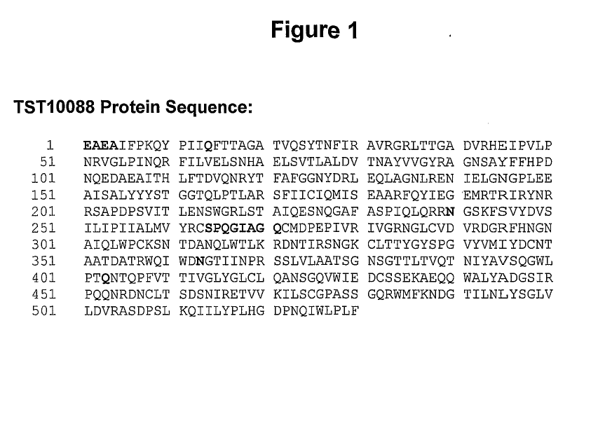

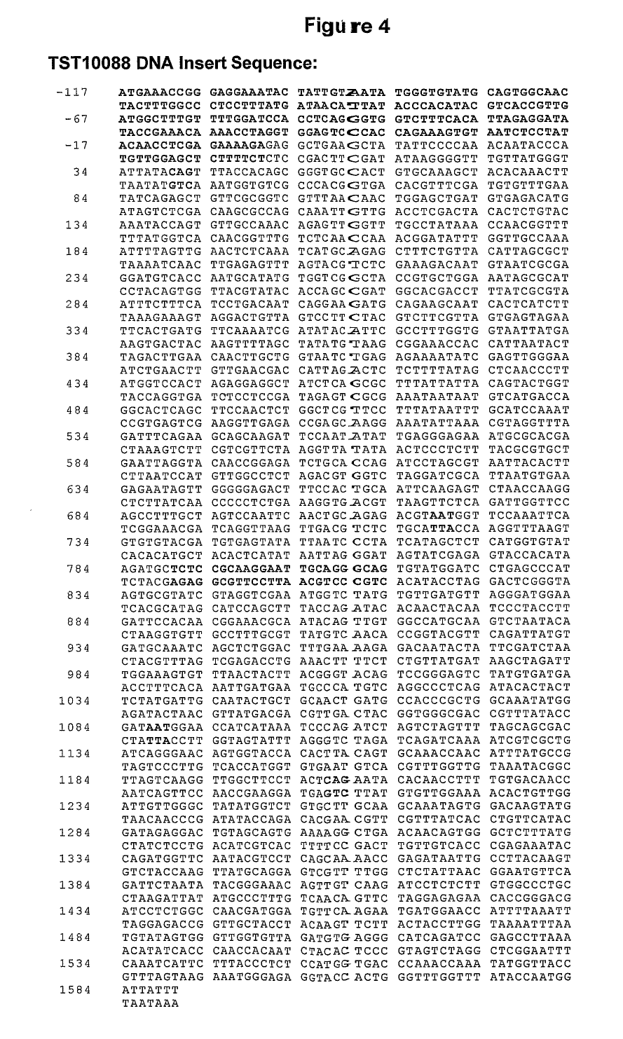

In the TST10088 construct (see FIGS. 1 and 4; SEQ ID Nos. 1 and 4, respectively), there are two sequons. In this construct, the inventors mutated the glycosylation sites at A1 and B2 leaving the glycosylation site at the A2 site (at amino acid position 240) and at the B1 site (at amino acid position 363). In the yeast expression system, the A2 site in the TST10088 construct is not glycosylated and thus the expressed protein is only glycosylated at a single site in the B-chain at B1.

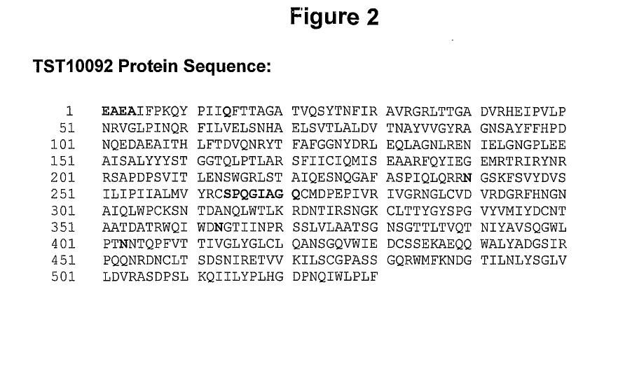

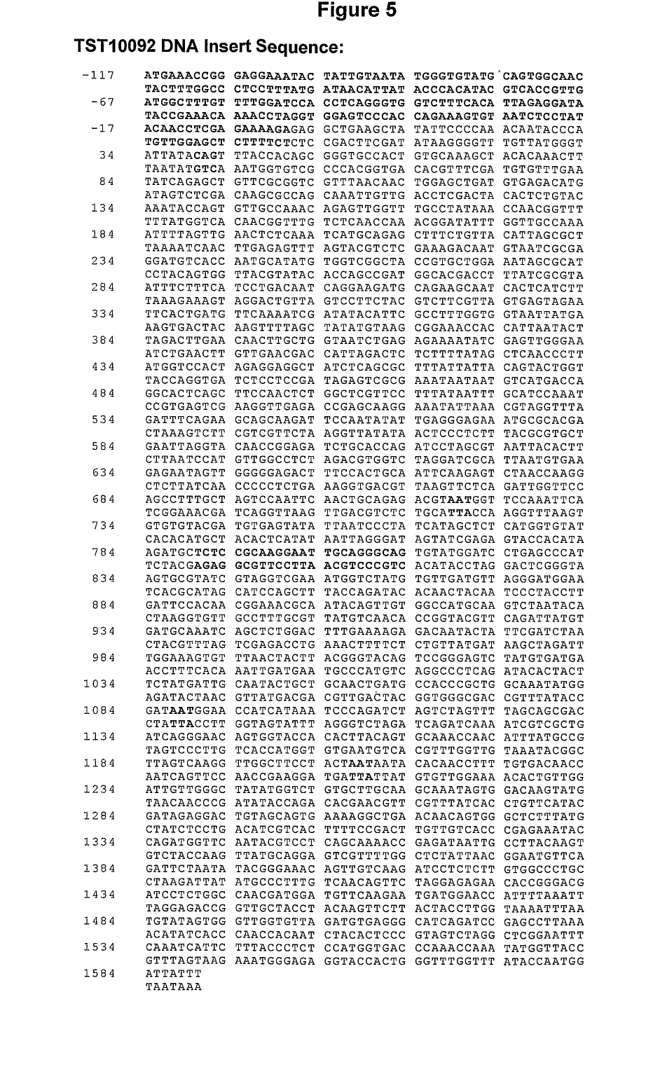

In the TST10092 construct (see FIGS. 2 and 5; SEQ ID Nos. 2 and 5, respectively), there are three sequons. In this construct, the inventors mutated the glycosylation site at A1 leaving the glycosylation site at the A2 site (at amino acid position 240), at the B1 site (at amino acid position 363) and at the B2 site (at amino acid position 403). In the yeast expression system, the A2 site in the TST10092 construct is not glycosylated, and thus the expressed protein is only glycosylated at two sites: B1 and B2.

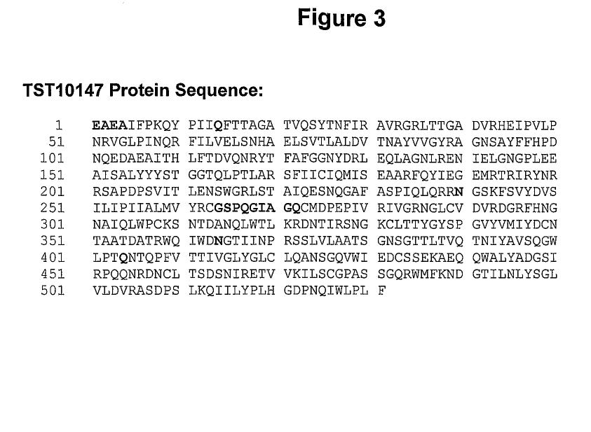

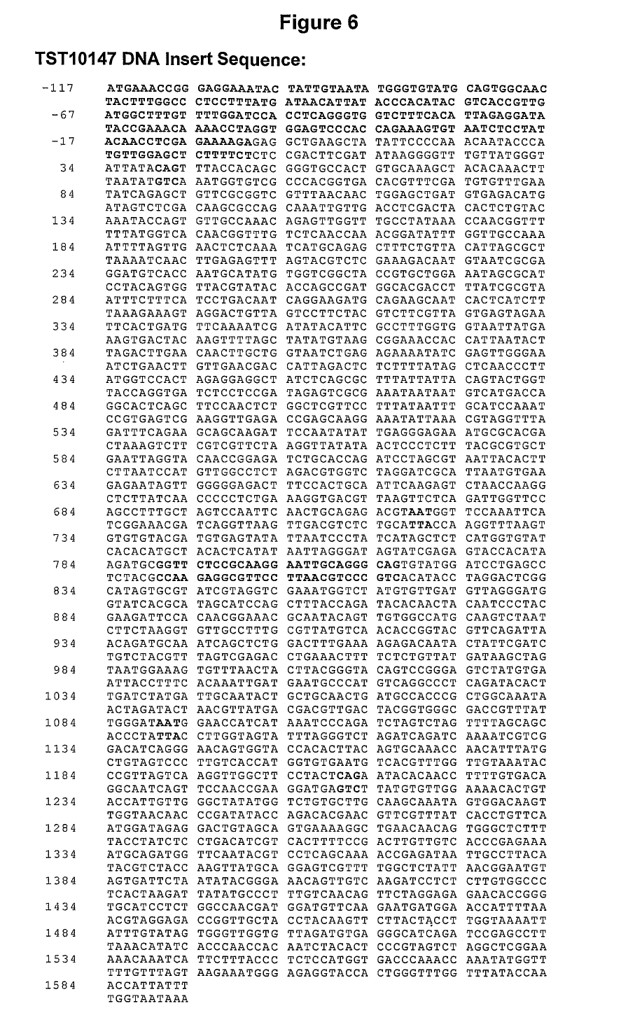

In the TST10147 construct (see FIGS. 3 and 6; SEQ ID Nos. 3 and 6, respectively), there are two sequons. In this construct, the inventors mutated the glycosylation sites at A1 and B2 leaving the glycosylation site at the A2 site (at amino acid position 240) and at the B1 site (at amino acid position 364). In the yeast expression system, the A2 site in the TST10147 construct is not glycosylated and thus the expressed protein is only glycosylated at a single site in the B-chain at B1.

Accordingly, the present invention provides a recombinant protein comprising (a) an A chain of a ricin-like toxin, (b) a B chain of a ricin-like toxin and (c) a heterologous linker amino acid sequence linking the A and B chains, the linker sequence containing a cleavage recognition site for a disease-specific protease, wherein the A chain or the B chain has at least one glycosylation site. In a preferred embodiment of the invention the B chain has at least one glycosylation site. In another preferred embodiment of the invention, the B chain is glycosylated at B1.

The term “glycosylation site” means an amino acid residue in the recombinant protein that can be glycosylated or linked to a carbohydrate. A “glycosylation site” can also be referred to as a “sequon” herein. Asn is an example of an amino acid that can be glycosylated. Serine and threonine are also examples of amino acids that can be glycosylated.

Preferably, the recombinant protein has been mutated in the A chain or B chain to block one or more glycosylation sites. Most preferably, the recombinant protein is only glycosylated at one site most preferably site B1.

The term “ricin-like toxins” includes, but is not limited to, bacterial, fungal and plant toxins which can inactivate ribosomes and inhibit protein synthesis. Most ricin-like toxins consist of an A-chain and a B-chain. The A chain is an active polypeptide subunit which is responsible for the pharmacologic effect of the toxin. In most cases the active component of the A chain is an enzyme. The B chain is responsible for binding the toxin to the cell surface and is thought to facilitate entry of the A chain into the cell cytoplasm. The A and B chains in the mature toxins are linked by disulfide bonds.

In a preferred embodiment, the ricin-like toxin is ricin. Ricin is a plant derived ribosome inhibiting protein which blocks protein synthesis in eukaryotic cells. Ricin may be derived from the seeds of Ricinus communis (castor oil plant). The ricin toxin is a glycosylated heterodimer with A and B chain molecular masses of 30,625 Da and 31,431 Da respectively. The A chain of ricin has an N-glycosidase activity and catalyzes the excision of a specific adenine residue from the 28S rRNA of eukaryotic ribosomes (Endo, Y; & Tsurugi, K. J. Biol. Chem. 262:8128 (1987)). The B chain of ricin, although not toxic in itself, promotes the toxicity of the A chain by binding to galactose residues on the surface of eukaryotic cells and stimulating receptor-mediated endocytosis of the toxin molecule (Simmons et al., Biol. Chem. 261:7912 (1986)).

The toxins most similar in structure to ricin are plant toxins which have one A chain and one B chain. Examples of such toxins include abrin which may be isolated from the seeds of Abrus precatorius and modeccin. Abrin has three glycosylation sites. One site is on the A-chain at position 203 (A1), and two sites are on the B-chain at positions 361 (B1) and 404 (B2). The A1 site does not appear to be glycosylated in plants, or in yeast expression systems.

Ricin-like bacterial toxins include diphtheria toxin, which is produced by Corynebacterium diphtheriae, Pseudomonas enterotoxin A and cholera toxin. It will be appreciated that the term “ricin-like toxin” is also intended to include the A chain of those toxins which have only an A chain. The recombinant proteins of the invention could include the A chain of these toxins conjugated to, or expressed as, a recombinant protein with the B chain of another toxin. Examples of plant toxins having only an A chain include trichosanthin, MMC and pokeweed antiviral proteins, dianthin 30, dianthin 32, crotin II, curcin II and wheat germ inhibitor. Examples of fungal toxins having only an A chain include alpha-sarcin, restrictocin, mitogillin, enomycin, phenomycin. Examples of bacterial toxins having only an A chain include cytotoxin from Shigella dysenteriae and related Shiga-like toxins. Recombinant trichosanthin and the coding sequence thereof is disclosed in U.S. Pat. Nos. 5,101,025 and 5,128,460.

In addition to the entire A or B chains of a ricin-like toxin, it will be appreciated that the recombinant protein of the invention may contain only that portion of the A chain which is necessary for exerting its cytotoxic effect. For example, the first 30 amino acids of the ricin A chain may be removed resulting in a truncated A chain which retains toxic activity. The truncated ricin or ricin-like A chain may be prepared by expression of a truncated gene or by proteolytic degradation, for example with Nagarase (Funmatsu et al., Jap. J. Med. Sci. Biol. 23:264-267 (1970)). Similarly, the recombinant protein of the invention may contain only that portion of the B chain necessary for galactose recognition, cell binding and transport into the cell cytoplasm. Truncated B chains are described for example in E.P. 145,111. It is appreciated that the ricin-like toxin may also have other modifications in the A chain or B chain that are immaterial and do not affect the activity of the protein. Such changes include analogs that function in the same way as the native A chain or B chain including analogs with conservative amino acid substitutions.

The term “linker sequence” as used herein refers to an internal amino acid sequence within the recombinant protein which contains residues linking the A and B chain so as to render the A chain incapable of exerting its toxic effect, for example catalytically inhibiting translation of a eukaryotic ribosome. By heterologous is meant that the linker sequence is not a sequence native to the A or B chain of a ricin-like toxin or precursor thereof. However, preferably, the linker sequence may be of a similar length to the linker sequence of a ricin-like toxin and should not interfere with the role of the B chain in cell binding and transport into the cytoplasm. When the linker sequence is cleaved the A chain becomes active or toxic.

The linker regions encode a cleavage recognition sequence for a disease-specific protease associated with for example, cancer, viruses, parasites, or fungi. The mutagenesis and cloning strategies used to generate a specific protease-sensitive linker variant are summarized in WO 9849311 to the present inventor. Briefly, the first step involves a DNA amplification using a set of mutagenic primers in combination with the two flanking primers Ricin-109Eco and Ricin1729C PstI. Restriction digested PCR fragments are gel purified and then ligated with PVL1393 which has been digested with Eco RI and PstI. Ligation reactions are used to transform competent XLI-Blue cells (Stratagene). Recombinant clones are identified by restriction digests of plasmid miniprep, DNA and the mutant linker sequences are confirmed by DNA sequencing. Specific linker sequences, which can be used with the present invention are detailed in U.S. Pat. Nos. 6,333,303; 6,531,125 and; 6,593,132.

The cleavage recognition sequence for a disease-associated protease in the linker chain can be a peptide mimetic. “Peptide mimetics” are structures which serve as substitutes for peptides in interactions between molecules (See Morgan et al (1989), Ann. Reports Med. Chem. 24:243-252 for a review). Peptide mimetics include synthetic structures which may or may not contain amino acids and/or peptide bonds but retain the structural and functional features of the cleavage recognition sequence in the linker chain. Peptide mimetics also include peptoids, oligopeptoids (Simon et al (1972) Proc. Natl. Acad, Sci USA 89:9367); and peptide libraries containing peptides of a designed length representing all possible sequences of amino acids corresponding to the cleavage recognition sequence of the invention

Peptide mimetics may be designed based on information obtained by systematic replacement of L-amino acids by D-amino acids, replacement of side chains with groups having different electronic properties, and by systematic replacement of peptide bonds with amide bond replacements. Local conformational constraints can also be introduced to determine conformational requirements for activity of a candidate peptide mimetic. The mimetics may include isosteric amide bonds, or D-amino acids to stabilize or promote reverse turn conformations and to help stabilize the molecule. Cyclic amino acid analogues may be used to constrain amino acid residues to particular conformational states. The mimetics can also include mimics of inhibitor peptide secondary structures. These structures can model the 3-dimensional orientation of amino acid residues into the known secondary conformations of proteins. Peptoids may also be used which are oligomers of N-substituted amino acids and can be used as motifs for the generation of chemically diverse libraries of novel molecules.

In a preferred embodiment of the invention, the recombinant protein has the amino acid sequence shown in FIG. 1, 2 or 3 (SEQ ID Nos. 1-3, respectively), or is a fragment or analog thereof. An analog of the referenced sequence will have a similar biological activity but may have differences in the amino acid sequences. Preferably, an analog will have conservative amino acid substitutions as compared to SEQ ID Nos. 1-3. Conserved amino acid substitutions involve replacing one or more amino acids of the recombinant proteins of the invention with amino acids of similar charge, size, and/or hydrophobicity characteristics. When only conserved substitutions are made the resulting analog should be functionally equivalent.

The inventors tested different secretion signals in an effort to improve expression levels. The term “secretion signal sequence” as used herein refers to an amino acid sequence which is required for the expression of a secretory protein. For example, the inventor tested the α-mating factor secretion signal from Saccharomyces cerevisiae, the Pho-1 secretion signal and the ricin secretion signal to drive protein expression and secretion. The best results were obtained using the natural ricin secretion signal. The inventor discovered that, in addition to improved overall protein yields, virtually all hyperglycosylation was eliminated when the gene was expressed using the ricin secretion signal. In an embodiment of the invention the recombinant protein of the invention has a secretion signal sequence that allows the expression of the recombinant protein without hyperglycosylation. In a preferred embodiment, the secretion signal sequence is the ricin secretion signal sequence.

The proteins of the invention may be prepared using recombinant DNA methods. Accordingly, the nucleic acid molecules of the present invention may be incorporated in a known manner into an appropriate expression vector which ensures good expression of the protein. Possible expression vectors include but are not limited to cosmids, plasmids, or modified viruses (e.g. replication defective retroviruses, adenoviruses and adeno-associated viruses), so long as the vector is compatible with the host cell used. The expression vectors are “suitable for transformation of a host cell”, which means that the expression vectors contain a nucleic acid molecule of the invention and regulatory sequences selected on the basis of the host cells to be used for expression, which is operatively linked to the nucleic acid molecule. Operatively linked is intended to mean that the nucleic acid is linked to regulatory sequences in a manner which allows expression of the nucleic acid.

The invention therefore contemplates a recombinant expression vector of the invention containing a nucleic acid molecule of the invention, or a fragment thereof, and the necessary regulatory sequences for the transcription and translation of the inserted protein-sequence.

Suitable regulatory sequences may be derived from a variety of sources, including bacterial, fungal, viral, mammalian, or insect genes (For example, see the regulatory sequences described in Goeddel, Gene Expression Technology Methods in Enzymology 185, Academic Press, San Diego, Calif. (1990). Selection of appropriate regulatory sequences is dependent on the host cell chosen as discussed below, and may be readily accomplished by one of ordinary skill in the art. Examples of such regulatory sequences include: a transcriptional promoter and enhancer or RNA polymerase binding sequence, a ribosomal binding sequence, including a translation initiation signal. Additionally, depending on the host cell chosen and the vector employed, other sequences, such as an origin of replication, additional DNA restriction sites, enhancers, and sequences conferring inducibility of transcription may be incorporated into the expression vector. It will also be appreciated that the necessary regulatory sequences may be supplied by the native A and B chains and/or its flanking regions.

The recombinant expression vectors of the invention may also contain a selectable marker gene which facilitates the selection of host cells transformed or transfected with a recombinant molecule of the invention. Examples of selectable marker genes are genes encoding a protein such as G418 and hygromycin which confer resistance to certain drugs, β-galactosidase, chloramphenicol acetyltransferase, firefly luciferase, or an immunoglobulin or portion thereof such as the Fc portion of an immunoglobulin preferably IgG. Transcription of the selectable marker gene is monitored by changes in the concentration of the selectable marker protein such as β-galactosidase, chloramphenicol acetyltransferase, or firefly luciferase. If the selectable marker gene encodes a protein conferring antibiotic resistance such as neomycin resistance transformant cells can be selected with G418. Cells that have incorporated the selectable marker gene will survive, while the other cells die. This makes it possible to visualize and assay for expression of recombinant expression vectors of the invention and in particular to determine the effect of a mutation on expression and phenotype. It will be appreciated that selectable markers can be introduced on a separate vector from the nucleic acid of interest.

The recombinant expression vectors may also contain genes which encode a fusion moiety which provides increased expression of the recombinant protein; increased solubility of the recombinant protein; and aid in the purification of the target recombinant protein by acting as a ligand in affinity purification. For example, a proteolytic cleavage site may be added to the target recombinant protein to allow separation of the recombinant protein from the fusion moiety subsequent to purification of the fusion protein. Typical fusion expression vectors include pGEX (Amrad Corp., Melbourne, Australia), pMal (New England Biolabs, Beverly, Mass.) and pRIT5 (Pharmacia, Piscataway, N.J.) which fuse glutathione S-transferase (GST), maltose E binding protein, or protein A, respectively, to the recombinant protein.

Recombinant expression vectors can be introduced into host cells to produce a transformed host cell. The term “transformed host cell” is intended to include cells that are capable of glycosylation which have been transformed or transfected with a recombinant expression vector of the invention. The terms “transformed with”, “transfected with”, “transformation” and “transfection” are intended to encompass introduction of nucleic acid (e.g. a vector) into a cell by one of many possible techniques known in the art. Prokaryotic cells can be transformed with nucleic acid by, for example, electroporation or calcium-chloride mediated transformation. For example, nucleic acid can be introduced into mammalian cells via conventional techniques such as calcium phosphate or calcium chloride co-precipitation, DEAE-dextran mediated transfection, lipofectin, electroporation or microinjection. Suitable methods for transforming and transfecting host cells can be found in Sambrook et al. (Molecular Cloning: A Laboratory Manual, 2nd Edition, Cold Spring Harbor Laboratory press (1989)), and other laboratory textbooks.

Suitable host cells include a wide variety of eukaryotic host cells and prokaryotic cells. For example, the proteins of the invention may be expressed in yeast cells or mammalian cells. Other suitable host cells can be found in Goeddel, Gene Expression Technology: Methods in Enzymology 185, Academic Press, San Diego, Calif. (1991). In addition, the proteins of the invention may be expressed in prokaryotic cells, such as Escherichia coli (Zhang et al., Science 303(5656): 371-3 (2004)).

Yeast and fungi host cells suitable for carrying out the present invention include, but are not limited to Saccharomyces cerevisiae, the genera Pichia or Kluyveromyces and various species of the genus Aspergillus. Examples of vectors for expression in yeast S. cerevisiae include pYepSec1 (Baldari. et al., Embo J. 6:229-234 (1987)), pMFa (Kurjan and Herskowitz, Cell 30:933-943 (1982)), pJRY88 (Schultz et al., Gene 54:113-123 (1987)), and pYES2 (Invitrogen Corporation, San Diego, Calif.). Protocols for the transformation of yeast and fungi are well known to those of ordinary skill in the art (see Hinnen et al., Proc. Natl. Acad. Sci. USA 75:1929 (1978); Itoh et al., J. Bacteriology 153:163 (1983), and Cullen et al. (Bio/Technology 5:369 (1987)).

In one embodiment of the invention, the recombinant protein of the invention is expressed in Pichia pastoris. The inventor found that glycosylation occurs at one position in the A-chain and the two sites in the B-chain when the natural A-chain and B-chain sequences of ricin are expressed in glycosylation-competent yeast. The second sequon in the A-chain appears to be inactive in yeast.

Mammalian cells suitable for carrying out the present invention include, among others: COS (e.g., ATCC No. CRL 1650 or 1651), BHK (e.g. ATCC No. CRL 6281), CHO (ATCC No. CCL 61), HeLa (e.g., ATCC No. CCL 2), 293 (ATCC No. 1573) and NS-1 cells. Suitable expression vectors for directing expression in mammalian cells generally include a promoter (e.g., derived from viral material such as polyoma, Adenovirus 2, cytomegalovirus and Simian Virus 40), as well as other transcriptional and translational control sequences. Examples of mammalian expression vectors include pCDM8 (Seed, B., Nature 329:840 (1987)) and pMT2PC (Kaufman et al., EMBO J. 6:187-195 (1987)).

Given the teachings provided herein, promoters, terminators, and methods for introducing expression vectors of an appropriate type into plant, avian, and insect cells may also be readily accomplished. For example, within one embodiment, the proteins of the invention may be expressed from plant cells (see Sinkar et al., J. Biosci (Bangalore) 11:47-58 (1987), which reviews the use of Agrobacterium rhizogenes vectors; see also Zambryski et al., Genetic Engineering, Principles and Methods, Hollaender and Setlow (eds.), Vol. VI, pp. 253-278, Plenum Press, New York (1984), which describes the use of expression vectors for plant cells, including, among others, PAPS2022, PAPS2023, and PAPS2034)

Insect cells suitable for carrying out the present invention include cells and cell lines from Bombyx, Trichoplusia or Spodotera species. Baculovirus vectors available for expression of proteins in cultured insect cells (SF 9 cells) include the pAc series (Smith et al., Mol. Cell. Biol. 3:2156-2165 (1983)) and the pVL series (Lucklow, V. A., and Summers, M. D., Virology 170:31-39 (1989)). Some baculovirus-insect cell expression systems suitable for expression of the recombinant proteins of the invention are described in PCT/US/02442.

Alternatively, the proteins of the invention may also be expressed in non-human transgenic animals such as, rats, rabbits, sheep and pigs (Hammer et al. Nature 315:680-683 (1985); Palmiter et al. Science 222:809-814 (1983); Brinster et al. Proc. Natl. Acad. Sci. USA 82:4438-4442 (1985); Palmiter and Brinster Cell 41:343-345 (1985) and U.S. Pat. No. 4,736,866).

The proteins of the invention may also be prepared by chemical synthesis using techniques well known in the chemistry of proteins such as solid phase synthesis (Merrifield, J. Am. Chem. Assoc. 85:2149-2154 (1964); Frische et al., J. Pept. Sci. 2(4): 212-22 (1996)) or synthesis in homogenous solution (Houbenweyl, Methods of Organic Chemistry, ed. E. Wansch, Vol. 15 I and II, Thieme, Stuttgart (1987)).

The present invention also provides proteins comprising an A chain of a ricin-like toxin, a B chain of a ricin-like toxin and a heterologous linker amino acid sequence linking the A and B chains, wherein the linker sequence contains a cleavage recognition site for a specific protease. Such a protein could be prepared other than by recombinant means, for example by chemical synthesis or by conjugation of A and B chains and a linker sequence isolated and purified from their natural plant, fungal or mammalian source.

N-terminal or C-terminal fusion proteins comprising the protein of the invention conjugated with other molecules, such as proteins may be prepared by fusing, through recombinant techniques. The resultant fusion proteins contain a protein of the invention fused to the selected protein or marker protein as described herein. The recombinant protein of the invention may also be conjugated to other proteins by known techniques. For example, the proteins may be coupled using heterobifunctional thiol-containing linkers as described in WO 90/10457, N-succinimidyl-3-(2-pyridyldithio-proprionate) or N-succinimidyl-5 thioacetate. Examples of proteins which may be used to prepare fusion proteins or conjugates include cell binding proteins such as immunoglobulins, hormones, growth factors, lectins, insulin, low density lipoprotein, glucagon, endorphins, transferrin, bombesin, asialoglycoprotein glutathione-S-transferase (GST), hemagglutinin (HA), and truncated myc.

### (B) Nucleic Acids of the Invention

The present invention relates to purified and isolated nucleic acid molecules comprising (a) a nucleotide sequence encoding an A chain of a ricin-like toxin, (b) a nucleotide sequence encoding a B chain of a ricin-like toxin and (c) a nucleotide sequence encoding a heterologous linker amino acid sequence linking the A and B chain, the heterologous linker sequence containing a cleavable recognition site for a disease-specific protease, wherein the nucleotide sequence encoding the A chain or the nucleotide sequence encoding the B chain encodes at least one amino acid having a glycosylation site. In a preferred embodiment of the invention, the nucleotide sequence of the B chain encodes at least one amino acid having a glycosylation site. In another preferred embodiment, the nucleotide sequence of the B chain encodes an amino acid at B1 having a glycosylation site.

In one embodiment of the invention, the nucleic acid molecule of the invention encodes a secretion signal sequence which allows expression of the recombinant protein of the invention, preferably without being hyperglycosylated. In a preferred embodiment, the secretion signal sequence is the ricin secretion signal sequence.

In another embodiment of the invention, the nucleic acid molecule of the invention has the nucleic acid sequence as shown in FIG. 4, 5 or 6 (SEQ ID Nos. 4-6, respectively).

In a preferred embodiment, the nucleic acid molecule comprises:
(a) a nucleic acid sequence as shown in FIG. 4 (SEQ. ID. NO.:4), FIG. 5 (SEQ. ID. NO.:5) or FIG. 6 (SEQ. ID. NO.:6) wherein T can also be U;
(b) a nucleic acid sequence that is complementary to a nucleic acid sequence of (a);
(c) a nucleic acid sequence that has substantial sequence homology to a nucleic acid sequence of (a) or (b);
(d) a nucleic acid sequence that is an analog of a nucleic acid sequence of (a), (b) or (c); or
(e) a nucleic acid sequence that hybridizes to a nucleic acid sequence of (a), (b), (c) or (d) under stringent hybridization conditions.

The term “sequence that has substantial sequence homology” means those nucleic acid sequences which have slight or inconsequential sequence variations from the sequences in (a) or (b), i.e., the sequences function in substantially the same manner. The variations may be attributable to local mutations or structural modifications. Nucleic acid sequences having substantial homology include nucleic acid sequences having at least 65%, more preferably at least 85%, and most preferably 90-95% identity with the nucleic acid sequences as shown in FIG. 4 (SEQ. ID. NO.:4), FIG. 5 (SEQ. ID. NO.:5), FIG. 6 (SEQ. ID. NO.:6)). Sequence identity can be calculated according to methods known in the art. Sequence identity is most preferably assessed by the algorithm of BLAST version 2.1 advanced search. BLAST is a series of programs that are available online at http://www.ncbi.nlm.nih.gov/BLAST. The advanced blast search (http://www.ncbi.nlm.nih.gov/blast/blast.cgi?Jform=1) is set to default parameters. (ie Matrix BLOSUM62; Gap existence cost 11; Per residue gap cost 1; Lambda ratio 0.85 default). References to BLAST searches are: Altschul, S. F., Gish, W., Miller, W., Myers, E. W. & Lipman, D. J. (1990) “Basic local alignment search tool.” J. Mol. Biol. 215:403-410; Gish, W. & States, D. J. (1993) “Identification of protein coding regions by database similarity search.” Nature Genet. 3:266272; Madden, T. L., Tatusov, R. L. & Zhang, J. (1996) “Applications of network BLAST server” Meth. Enzymol. 266:131_{—}141; Altschul, S. F., Madden, T. L., Schäffer, A. A., Zhang, J., Zhang, Z., Miller, W. & Lipman, D. J. (1997) “Gapped BLAST and PSI_BLAST: a new generation of protein database search programs.” Nucleic Acids Res. 25:33893402; Zhang, J. & Madden, T. L. (1997) “PowerBLAST: A new network BLAST application for interactive or automated sequence analysis and annotation.” Genome Res. 7:649656.

The term “sequence that hybridizes” means a nucleic acid sequence that can hybridize to a sequence of (a), (b), (c) or (d) under stringent hybridization conditions. Appropriate “stringent hybridization conditions” which promote DNA hybridization are known to those skilled in the art, or may be found in Current Protocols in Molecular Biology, John Wiley & Sons, N.Y. (1989), 6.3.1-6.3.6. The term “stringent hybridization conditions” as used herein means that conditions are selected which promote selective hybridization between two complementary nucleic acid molecules in solution. Hybridization may occur to all or a portion of a nucleic acid sequence molecule. The hybridizing portion is at least 50% the length with respect to one of the polynucleotide sequences encoding a polypeptide. In this regard, the stability of a nucleic acid duplex, or hybrids, is determined by the Tm, which in sodium containing buffers is a function of the sodium ion concentration, G/C content of labeled nucleic acid, length of nucleic acid probe (I), and temperature (Tm=81.5° C.−16.6(Log 10[Na+])+0.41(%(G+C)−600/1). Accordingly, the parameters in the wash conditions that determine hybrid stability are sodium ion concentration and temperature. In order to identify molecules that are similar, but not identical, to a known nucleic acid molecule a 1% mismatch may be assumed to result in about a 1° C. decrease in Tm, for example if nucleic acid molecules are sought that have a greater than 95% identity, the final wash will be reduced by 5° C. Based on these considerations stringent hybridization conditions shall be defined as: hybridization at 5× sodium chloride/sodium citrate (SSC)/5×Denhardt's solution/1.0% SDS at Tm (based on the above equation)-5° C., followed by a wash of 0.2×SSC/0.1% SDS at 60° C.

The term “a nucleic acid sequence which is an analog” means a nucleic acid sequence which has been modified as compared to the sequence of (a), (b) or (c) wherein the modification does not alter the utility of the sequence as described herein. The modified sequence or analog may have improved properties over the sequence shown in (a), (b) or (c). One example of a modification to prepare an analog is to replace one of the naturally occurring bases (i.e. adenine, guanine, cytosine or thymidine) of the sequence shown in FIG. 4 (SEQ ID NO:4), FIG. 5 (SEQ ID NO:5) or FIG. 6 (SEQ ID NO:6) with a modified base such as such as xanthine, hypoxanthine, 2-aminoadenine, 6-methyl, 2-propyl and other alkyl adenines, 5-halo uracil, 5-halo cytosine, 6-aza uracil, 6-aza cytosine and 6-aza thymine, pseudo uracil, 4-thiouracil, 8-halo adenine, 8-aminoadenine, 8-thiol adenine, 8-thiolalkyl adenines, 8-hydroxyl adenine and other 8-substituted adenines, 8-halo guanines, 8 amino guanine, 8-thiol guanine, 8-thiolalkyl guanines, 8-hydroxyl guanine and other 8-substituted guanines, other aza and deaza uracils, thymidines, cytosines, adenines, or guanines, 5-trifluoromethyl uracil and 5-trifluoro cytosine.

Another example of a modification is to include modified phosphorous or oxygen heteroatoms in the phosphate backbone, short chain alkyl or cycloalkyl intersugar linkages or short chain heteroatomic or heterocyclic intersugar linkages in the nucleic acid molecule shown in FIG. 4 (SEQ ID NO:4), FIG. 5 (SEQ ID NO:5) or FIG. 6 (SEQ ID NO:6). For example, the nucleic acid sequences may contain phosphorothioates, phosphotriesters, methyl phosphonates, and phosphorodithioates.

A further example of an analog of a nucleic acid molecule of the invention is a peptide nucleic acid (PNA) wherein the deoxyribose (or ribose) phosphate backbone in the DNA (or RNA), is replaced with a polyamide backbone which is similar to that found in peptides (P. E. Nielsen, et al Science 1991, 254, 1497). PNA analogs have been shown to be resistant to degradation by enzymes and to have extended lives in vivo and in vitro. PNAs also bind stronger to a complimentary DNA sequence due to the lack of charge repulsion between the PNA strand and the DNA strand. Other nucleic acid analogs may contain nucleotides containing polymer backbones, cyclic backbones, or acyclic backbones. For example, the nucleotides may have morpholino backbone structures (U.S. Pat. No. 5,034,506). The analogs may also contain groups such as reporter groups, a group for improving the pharmacokinetic or pharmacodynamic properties of nucleic acid sequence.

The term “isolated and purified” as used herein refers to a nucleic acid substantially free of cellular material or culture medium when produced by recombinant DNA techniques, or chemical precursors, or other chemicals when chemically synthesized. An “isolated and purified” nucleic acid is also substantially free of sequences which naturally flank the nucleic acid (i.e. sequences located at the 5′ and 3′ ends of the nucleic acid) from which the nucleic acid is derived. The term “nucleic acid” is intended to include DNA and RNA and can be either double stranded or single stranded.

The nucleic acid molecule of the invention encoding a recombinant toxic protein is cloned by subjecting a preproricin cDNA clone to site-directed mutagenesis in order to generate a series of glycosylation variants. Oligonucleotides, corresponding to the extreme 5′ and 3′ ends of the preproricin gene are synthesized and used to PCR amplify the gene. Using the cDNA sequence for preproricin (Lamb et al., Eur. J. Biochem. 145:266-270 (1985)), several oligonucleotide primers are designed to flank the start and stop codons of the preproricin open reading frame.

The preproricin cDNA is amplified using the upstream primer Ricin-99 or Ricin-109 and the downstream primer Ricin1729C with Vent DNA polymerase (New England Biolabs) using standard procedures (Sambrook et al., Molecular Cloning: A Laboratory Manual, Second Edition, (Cold Spring Harbor Laboratory Press, 1989)). The purified PCR fragment encoding the preproricin cDNA is, then ligated into an Eco RV-digested pBluescript II SK plasmid (Stratagene), and is used to transform competent XL1-Blue cells (Stratagene). The cloned PCR product containing the putative preproricin gene is confirmed by DNA sequencing of the entire cDNA clone.

The preproricin cDNA clone is subjected to Quickchange mutagenesis (Stratagene); in order to generate a series of glycosylation variants. In a specific embodiment, the mutation involves replacing the nucleic acid sequence encoding Asn with a nucleic acid sequence encoding Gln in one or more sequons.

As mentioned above, the ricin gene has been cloned and sequenced, and the X-ray crystal structures of the A and B chains are published. It will be appreciated that the invention includes nucleic acid molecules encoding truncations of A and B chains of ricin like proteins and analogs and homologs of A and B chains of ricin-like proteins and truncations thereof (i.e., ricin-like proteins), as described herein. It will further be appreciated that variant forms of the nucleic acid molecules of the invention which arise by alternative splicing of an mRNA corresponding to cDNA of the invention are encompassed by the invention.

The nucleic acid molecule may comprise the A and/or B chain of a ricin-like toxin. Methods for cloning ricin-like toxins are known in the art and are described, for example, in E.P. 466,222. Sequences encoding ricin or ricin-like A and B chains may be obtained by selective amplification of a coding region, using sets of degenerative primers or probes for selectively amplifying the coding region in a genomic or cDNA library. Appropriate primers may be selected from the nucleic acid sequence of A and B chains of ricin or ricin-like toxins. It is also possible to design synthetic oligonucleotide primers from the nucleotide sequences for use in PCR. Suitable primers may be selected from the sequences encoding regions of ricin-like proteins which are highly conserved, as described for example in U.S. Pat. No. 5,101,025 and E.P. 466,222.

A nucleic acid can be amplified from cDNA or genomic DNA using these oligonucleotide primers and standard PCR amplification techniques. The nucleic acid so amplified can be cloned into an appropriate vector and characterized by DNA sequence analysis. It will be appreciated that cDNA may be prepared from mRNA, by isolating total cellular mRNA by a variety of techniques, for example, by using the guanidinium-thiocyanate extraction procedure of Chirgwin et al. (Biochemistry 18, 5294-5299 (1979)). cDNA is then synthesized from the mRNA using reverse transcriptase (for example, Moloney MLV reverse transcriptase available from Gibco/BRL, Bethesda, Md., or AMV reverse transcriptase available from Seikagaku America, Inc., St. Petersburg, Fla.). It will be appreciated that the methods described above may be used to obtain the coding sequence from plants, bacteria or fungi, preferably plants, which produce known ricin-like proteins and also to screen for the presence of genes encoding as yet unknown ricin-like proteins.

A sequence containing a cleavage recognition site for a specific protease may be selected based on the disease or condition which is to be targeted by the recombinant protein. The cleavage recognition site may be selected from sequences known to encode a cleavage recognition site specific proteases of the disease or condition to be treated. Sequences encoding cleavage recognition sites may be identified by testing the expression product of the sequence for susceptibility to cleavage by the respective protease. A polypeptide containing the suspected cleavage recognition site may be incubated with a specific protease and the amount of cleavage product determined (Dilannit, 1990, J. Biol. Chem. 285: 17345-17354 (1990)). The specific protease may be prepared by methods known in the art and used to test suspected cleavage recognition sites.

The nucleic acid molecule of the invention may also encode a fusion protein. A sequence encoding a heterologous linker sequence containing a cleavage recognition site for a specific protease may be cloned from a cDNA or genomic library or chemically synthesized based on the known sequence of such cleavage sites. The heterologous linker sequence may then be fused in frame with the sequences encoding the A and B chains of the ricin-like toxin for expression as a fusion protein. It will be appreciated that a nucleic acid molecule encoding a fusion protein may contain a sequence encoding an A chain and a B chain from the same ricin-like toxin or the encoded A and B chains may be from different toxins. For example, the A chain may be derived from ricin and the B chain may be derived from abrin. A protein may also be prepared by chemical conjugation of the A and B chains and linker sequence using conventional coupling agents for covalent attachment.

An isolated and purified nucleic acid molecule of the invention which is RNA can be isolated by cloning a cDNA encoding an A and B chain and a linker into an appropriate vector which allows for transcription of the cDNA to produce an RNA molecule which encodes a protein of the invention. For example, a cDNA can be cloned downstream of a bacteriophage promoter, (e.g. a T7 promoter) in a vector, cDNA can be transcribed in vitro with T7 polymerase, and the resultant RNA can be isolated by standard techniques.

### (C) Utility of the Recombinant Proteins and Nucleic Acid Molecules of the Invention

### (i) Therapeutic Methods

In one embodiment, the invention provides a method of inhibiting or destroying cells affected by a disease, which cells are associated with a protease specific to the disease comprising the steps of: (a) preparing a purified isolated nucleic acid of the invention; (b) introducing the nucleic acid into a host cell and expressing the nucleic acid in the host cell to obtain a recombinant protein of the invention; (c) suspending the protein in a pharmaceutically acceptable carrier, diluent or excipient; and (d) contacting the cells with the recombinant protein.

The terms “nucleic acid of the invention” and “recombinant protein of the invention” are used for ease of referral and include all of the nucleic acid molecules and recombinant proteins referred to in Section A and B as well as in the Examples and Figures.

In another embodiment, the invention provides a method of inhibiting or destroying cells affected by a disease comprising the steps of contacting the cells with the recombinant protein of the invention. The present invention also includes a use of a recombinant protein of the invention to inhibit or destroy cells affected by a disease. The invention further includes a use of a recombinant protein of the invention in the manufacture of a medicament to inhibit or destroy cells affected by a disease.

Matrix metalloproteinases (MMPs or matrixins) are zinc-dependent proteinases and the expression of MMP genes is reported to be activated in inflammatory disorders (e.g. rheumatoid arthritis) and malignancy. In addition, there are reports of increased activation and expression of urokinase type plasminogen activator in inflammatory disorders such a rheumatoid arthritis (Slot, O., et al. 1999), osteoarthritis (Pap, G. et al., 2000), atherosclerotic cells (Falkenberg, M., et al., 1998) Crohn's disease (Desreumaux P, et al. 1999), central nervous system disease (Cuzner and Opdenakker, 1999) as well as in malignancy. Accordingly, the recombinant proteins of the invention may be used to specifically inhibit or destroy cells affected by a disease.

The term “cells affected by a disease” refers to cells affected by a disease or infection, which have associated with such cells a specific protease that can cleave a linker sequence of the recombinant protein, for example, cancer cells, inflammatory cells, or cells infected with a virus, a fungus or a parasite. Disease includes various forms of cancer such as such as T- and B-cell lymphoproliferative diseases, ovarian cancer, pancreatic cancer, head and neck cancer, squamous cell carcinoma, gastrointestinal cancer, breast cancer, prostate cancer, lung cancer, and liver cancer. Disease also includes malaria, and diverse viral disease states associated with infection such as human cytomegalovirus, hepatitis virus, herpes virus, human rhinovirus, human T-cell leukemia virus, infectious laryngotracheitis virus, poliomyelitis virus, or varicella zoster virus. Disease also includes parasitic infections, such as with the parasite Plasmodium falciparum.

More particularly, the recombinant proteins of the invention may be used to specifically inhibit or destroy cancer cells that contain a protease that can cleave the linker sequence of the recombinant protein.

It is an advantage of the recombinant proteins of the invention that they have specificity for cells that contain a specific protease, including those of inflammatory disorders and cancer cells, without the need for a cell binding component. The ricin-like B chain of the recombinant proteins recognize galactose moieties on the cell surface and ensure that the protein is taken up by, for example, a cancer cell and released into the cytoplasm. When the protein is internalized into a normal cell, cleavage of the heterologous linker would not occur in the absence of the specific protease, and the A chain will remain inactive bound to the B chain. Conversely, when the protein is internalized into a cell having a specific protease, the specific protease will cleave the cleavage recognition site in the linker thereby releasing the toxic A chain.

Accordingly, the present invention provides a method of inhibiting or destroying cells having a specific protease, for examples inflammatory cells or cancer cells, comprising contacting such cells with an effective amount of a recombinant protein or a nucleic acid molecule encoding a recombinant protein of the invention. The present invention also provides a method of treating a cell having a specific protease, comprising administering an effective amount of a recombinant protein or a nucleic acid molecule encoding a recombinant protein of the invention to an animal in need thereof. The invention also includes a use of an effective amount of a recombinant protein or a nucleic acid molecule encoding a recombinant protein of the invention to treat a cell having a specific protease. The invention further includes a use of an effective amount of a recombinant protein or a nucleic acid molecule encoding a recombinant protein of the invention in the manufacture of a medicament to treat a cell having a specific protease.

The term “effective amount” as used herein means an amount effective, at dosages and for periods of time necessary to achieve the desired result.

The term “animal” as used herein means any member of the animal kingdom including all mammals, birds, fish, reptiles and amphibians. Preferably, the animal to be treated is a mammal, more preferably a human.

The term “treatment or treating” as used herein means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treating” can also mean prolonging survival as compared to expected survival if not receiving treatment.

The specificity of a recombinant protein of the invention may be tested by treating the protein with the specific protease which is thought to be specific for the cleavage recognition site of the linker and assaying for cleavage products. For example, specific proteases may be isolated from cancer cells, or they may be prepared recombinantly, for example following the procedures in Darket et al. (J. Biol. Chem. 254:2307-2312 (1988)). The cleavage products may be identified for example based on size, antigenicity or activity. The toxicity of the recombinant protein may be investigated by subjecting the cleavage products to an in vitro translation assay in cell lysates, for example using Brome Mosaic Virus mRNA as a template. Toxicity of the cleavage products may be determined using a ribosomal inactivation assay (Westby et al., Bioconjugate Chem. 3:377-382 (1992)). The effect of the cleavage products on protein synthesis may be measured in standardized assays of in vitro translation utilizing partially defined cell free systems composed for example of a reticulocyte lysate preparation as a source of ribosomes and various essential cofactors, such as mRNA template and amino acids. Use of radiolabelled amino acids in the mixture allows quantitation of incorporation of free amino acid precursors into trichloroacetic acid precipitable proteins. Rabbit reticulocyte lysates may be conveniently used (O'Hare, FEBS Lett. 273:200-204 (1990)).

The ability of the recombinant proteins of the invention to selectively inhibit or destroy cells having specific proteases may be readily tested in vitro using cell lines having the specific protease, such as cancer cell lines. The selective inhibitory effect of the recombinant proteins of the invention may be determined, for example, by demonstrating the selective inhibition of cellular proliferation in cancer cells or infected cells.

Toxicity may also be measured based on cell viability, for example, the viability of cancer and normal cell cultures exposed to the recombinant protein may be compared. Cell viability may be assessed by known techniques, such as trypan blue exclusion assays.

In another example, a number of models may be used to test the cytotoxicity of recombinant proteins having a heterologous linker sequence containing a cleavage recognition site for a cancer associated matrix metalloprotease. Thompson, E. W. et al. (Breast Cancer Res. Treatment 31:357-370 (1994)) has described a model for the determination of invasiveness of human breast cancer cells in vitro by measuring tumour cell-mediated proteolysis of extracellular matrix and tumour cell invasion of reconstituted basement membrane (collagen, laminin, fibronectin, Matrigel or gelatin). Other applicable cancer cell models include cultured ovarian adenocarcinoma cells (Young, T. N. et al. Gynecol. Oncol. 62:89-99 (1996); Moore, D. H. et al. Gynecol. Oncol. 65:78-82 (1997)), human follicular thyroid cancer cells (Demeure, M. J. et al., World J. Surg. 16:770-776 (1992)), human melanoma (A-2058) and fibrosarcoma (HT-1080) cell lines (Mackay, A. R. et al. Lab. Invest. 70:781 783 (1994)), and lung squamous (HS-24) and adenocarcinoma (SB-3) cell lines (Spiess, E. et al. J. Histochem. Cytochem. 42:917-929 (1994)). An in vivo test system involving the implantation of tumours and measurement of tumour growth and metastasis in athymic nude mice has also been described (Thompson, E. W. et al., Breast Cancer Res. Treatment 31:357-370 (1994); Shi, Y. E. et al., Cancer Res. 53:1409-1415 (1993)).

Although the primary specificity of the proteins of the invention for cells having a specific protease is mediated by the specific cleavage of the cleavage recognition site of the linker, it will be appreciated that specific cell binding components may optionally be conjugated to the proteins of the invention. Such cell binding components may be expressed as fusion proteins with the proteins of the invention or the cell binding component may be physically or chemically coupled to the protein component. Examples of suitable cell binding components include antibodies to cancer proteins, cytokines and receptor fragments (Frankel et al., Protein Eng. 9(10): 913-9 (1996); Frankel et al., Carbohydr. Res. 300(3): 251-8 (1997)).

Antibodies having specificity for a cell surface protein may be prepared by conventional methods. A mammal, (e.g. a mouse, hamster, or rabbit) can be immunized with an immunogenic form of the peptide which elicits an antibody response in the mammal. Techniques for conferring immunogenicity on a peptide include conjugation to carriers or other techniques well known in the art. For example, the peptide can be administered in the presence of adjuvant. The progress of immunization can be monitored by detection of antibody titers in plasma or serum. Standard ELISA or other immunoassay procedures can be used with the immunogen as antigen to assess the levels of antibodies. Following immunization, antisera can be obtained and, if desired, polyclonal antibodies isolated from the sera.

To produce monoclonal antibodies, antibody producing cells (lymphocytes) can be harvested from an immunized animal and fused with myeloma cells by standard somatic cell fusion procedures thus immortalizing these cells and yielding hybridoma cells. Such techniques are well known in the art, (e.g. the hybridoma technique originally developed by Kohler and Milstein (Nature 256:495-497 (1975)) as well as other techniques such as the human B-cell hybridoma technique (Kozbor et al., Immunol. Today 4:72 (1983)), the EBV-hybridoma technique to produce human monoclonal antibodies (Cole et al., Methods Enzymol, 121:140-67 (1986)), and screening of combinatorial antibody libraries (Huse et al., Science 246:1275 (1989)). Hybridoma cells can be screened immunochemically for production of antibodies specifically reactive with the peptide and the monoclonal antibodies can be isolated.

The term “antibody” as used herein is intended to include fragments thereof which also specifically react with a cell surface component. Antibodies can be fragmented using conventional techniques and the fragments screened for utility in the same manner as described above. For example, F(ab′)2 fragments can be generated by treating antibody with pepsin. The resulting F(ab′)2 fragment can be treated to reduce disulfide bridges to produce Fab fragments.

Chimeric antibody derivatives, i.e., antibody molecules that combine a non-human animal variable region and a human constant region are also contemplated within the scope of the invention. Chimeric antibody molecules can include, for example, the antigen binding domain from an antibody of a mouse, rat, or other species, with human constant regions. Conventional methods may be used to make chimeric antibodies containing the immunoglobulin variable region which recognizes a cell surface antigen (See, for example, Morrison et al., Proc. Natl Acad. Sci. U.S.A. 81:6851 (1985); Takeda et al., Nature 314:452 (1985), Cabilly et al., U.S. Pat. No. 4,816,567; Boss et al., U.S. Pat. No. 4,816,397; Tanaguchi et al., E.P. Patent No. 171,496; European Patent No. 173,494; United Kingdom Patent No. GB 2177096B). It is expected that chimeric antibodies would be less immunogenic in a human subject than the corresponding non-chimeric antibody.

Monoclonal or chimeric antibodies specifically reactive against cell surface components can be further humanized by producing human constant region chimeras, in which parts of the variable regions, particularly the conserved framework regions of the antigen-binding domain, are of human origin and only the hypervariable regions are of non-human origin. Such immunoglobulin molecules may be made by techniques known in the art, (e.g. Teng et al., Proc. Natl. Acad. Sci. U.S.A., 80:7308-7312 (1983); Kozbor et al., Immunology Today 4:7279 (1983); Olsson et al., Meth. Enzymol., 92:3-16 (1982), and PCT Publication WO92/06193 or EP 239,400). Humanized antibodies can also be commercially produced (Scotgen Limited, 2 Holly Road, Twickenham, Middlesex, Great Britain.)

Specific antibodies, or antibody fragments, reactive against cell surface components may also be generated by screening expression libraries encoding immunoglobulin genes, or portions thereof, expressed in bacteria with cell surface components. For example, complete Fab fragments, VH regions and FV regions can be expressed in bacteria using phage expression libraries (See for example Ward et al., Nature 341:544-546 (1989); Huse et al., Science 246:1275-1281 (1989); and McCafferty et al., Nature 348:552-554 (1990)). Alternatively, a SCID-hu mouse, for example the model developed by Genpharm, can be used to produce antibodies, or fragments thereof.

### (ii) Pharmaceutical Compositions

The proteins and nucleic acids of the invention may be formulated into pharmaceutical compositions for administration to subjects in a biologically compatible form suitable for administration in vivo. By “biologically compatible form suitable for administration in vivo” is meant a form of the substance to be administered in which any toxic effects are outweighed by the therapeutic effects. The substances may be administered to living organisms including humans, and animals. Administration of a therapeutically active amount of the pharmaceutical compositions of the present invention is defined as an amount effective, at dosages and for periods of time necessary to achieve the desired result. For example, a therapeutically active amount of a substance may vary according to factors such as the disease state, age, sex, and weight of the individual, and the ability of the recombinant protein of the invention to elicit a desired response in the individual. Dosage regime may be adjusted to provide the optimum therapeutic response. For example, several divided doses may be administered daily or the dose may be proportionally reduced as indicated by the exigencies of the therapeutic situation.

Accordingly, the present invention provides a pharmaceutical composition for treating cells having a specific protease comprising a recombinant protein or a nucleic acid encoding a recombinant protein of the invention and a pharmaceutically acceptable carrier, diluent or excipient.

The active substance may be administered in a convenient manner such as by injection (subcutaneous, intravenous, intramuscular, etc.), oral administration, inhalation, transdermal administration (such as topical cream or ointment, etc.), or suppository applications. Depending on the route of administration, the active substance may be coated in a material to protect the compound from the action of enzymes, acids and other natural conditions which may inactivate the compound.

The compositions described herein can be prepared by per se known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985). On this basis, the compositions include, albeit not exclusively, solutions of the substances in association with one or more pharmaceutically acceptable vehicles or diluents, and contained in buffered solutions with a suitable pH and iso-osmotic with the physiological fluids.

The pharmaceutical compositions may be used in methods for treating animals, including mammals, preferably humans, with cancer or infected with a virus, a fungus or a parasite. The dosage and type of recombinant protein to be administered will depend on a variety of factors which may be readily monitored in human subjects. Such factors include the etiology and severity (grade and stage) of the neoplasia or infection.

(iii) Combination Therapies

In the majority of approved anticancer therapy, drugs are used in combination. The inventors found that the recombinant proteins of the invention had supradditive activity when used in combination with other conventional anticancer therapies. The term “anticancer therapy” includes any anticancer therapy including, without limitation, chemotherapeutic agents such as doxorubicin, cisplatin, cyclophosphamide etoposide, paclitaxel, taxotere, carboplatin, oxaliplatin, 5-fluorouracil, irinotecan, topotecan, vincristine, gemcitabine, epirubicin, capecitabine, and temozolomide.

In an embodiment, the invention provides a method of inhibiting or destroying cells affected by cancer using the recombinant proteins and nucleic acids of the invention in combination with at least one other anticancer therapy. The invention also includes a use of a) a recombinant protein or nucleic acid of the invention in combination with b) an additional cancer therapy to inhibit or destroy cells affected by cancer. The present invention further includes a use of a) a recombinant protein or nucleic acid of the invention in combination with b) an additional anticancer therapy in the manufacture of a medicament to inhibit or destroy cells affected by cancer.

In another embodiment, the invention provides a method of treating a mammal with cancer comprising the steps of preparing the recombinant protein of the invention and administering the protein to the mammal in combination with at least one other anticancer therapy.

Another embodiment of the invention is a process for preparing a pharmaceutical for treating a mammal with cancer using the recombinant proteins of the invention and/or the nucleic acids of the invention, and at least one other anticancer therapy. A further embodiment of the invention is a pharmaceutical composition for treating cancer which has the recombinant proteins of the invention and/or the nucleic acids of the invention, and at least one other anticancer therapy.

The following non-limiting examples are illustrative of the present invention:

### EXAMPLES

### Example 1

### Glycosylation Variants

Natural Ricin is a glycoprotein possessing N-linked carbohydrate. According to the amino acid sequence of ricin there are four sequons: two sites in the A-chain and two sites in the B-chain. There are also four sequons in the ricin-derived prodrugs produced by the inventors. The two sequons on the A-chain are referred to as A1 and A2; while the two on the B-chain are referred to as B1 and B2. In the proricin construct with an 8 amino acid linker, the A1 glycosylation site is at amino acid position 14, the A2 glycosylation site is at amino acid position 240, the B1 glycosylation site is at amino acid position 363 and the B2 glycosylation site is at amino acid position 403. To some extent glycosylation occurs at all four sites in the natural protein—although the importance of glycosylation to the stability and activity of the molecule is not entirely clear. The inventors use a glycosylation-competent yeast to produce the ricin-derived prodrugs. The Applicant observed that glycosylation occurs at only one position in the A-chain and the two sites in the B-chain when yeast is used as the expression for prodrugs TST10001 through TST10007. Apparently, the second sequon in the A-chain is inactive in yeast.

Recombinant glycoproteins are problematic because they tend to be heterogeneous in their carbohydrate component. Moreover, variations in the fermentation process used to generate a recombinant may influence the character of this heterogeneity—i.e., heterogeneity can be manifest by variation in the number of carbohydrate chains attached to the protein or in differences in the composition of individual chains, or both.

The inventors examined 32 glycosylation variants (Tables 1 and 2) where sequons were modified or removed.

In the TST10088 construct (see FIGS. 1 and 4 (SEQ ID Nos. 1 and 4, respectively), there are two sequons (A2 and B1). In the yeast expression system, the A2 site in the TST10088 construct is not glycosylated, and thus the expressed protein is only glycosylated at a single site in the B-chain at B1.

FIG. 1 shows the amino acid sequence of the TST10088 construct (SEQ ID No. 1). The residual KEX2 cleavage amino acids (Glu-Ala-Glu-Ala) are designated in bold (amino acid positions 1 to 4). The A1 glycosylation site was mutated from Asn to Gln and is designated in bold (amino acid position 14). The A2 glycosylation site is designated in bold (amino acid position 240). The B1 glycosylation site is designated in bold (amino acid position 363). The B2 glycosylation site was mutated from Asn to Gln and is designated in bold (amino acid position 403). The linker sequence amino acids are designated in bold (amino acid positions 264 to 271).

FIG. 4 shows the nucleic acid sequence of the TST10088 construct (SEQ ID No. 4). The native proricin secretion signal is designated in bold (nucleotide positions −117 to −13). The A1 glycosylation site was mutated from Asn to Gln and is designated in bold (nucleotide positions 40 to 42). The A1 glycosylation site is designated in bold (nucleotide positions 718 to 720). The B1 glycosylation site is designated in bold (nucleotide positions 1087 to 1089). The B2 glycosylation site was mutated from Asn to Gln and is designated in bold (nucleotide positions 1207 to 1209). The linker sequence is designated in bold (nucleotide positions 790 to 813). The KEX-2 cleavage signal is designated in bold (nucleotide positions −13 to −1).

In the TST10092 construct (see FIGS. 2 and 5), there are three sequons (A2, B1 and B2). In the yeast expression system, the A2 site in the TST10092 construct is not glycosylated, and thus the expressed protein is only glycosylated at two sites (B1 and B2).

FIG. 2 shows the amino acid sequence of the TST10092 construct (SEQ ID No. 2). The residual KEX2 cleavage amino acid (Glu-Ala-Glu-Ala) are designated in bold (amino acid positions 1 to 4). The A1 glycosylation site was mutated from Asn to Gln and is designated in bold (amino acid position 14). The A2 glycosylation site is designated in bold (amino acid position 240). The B1 glycosylation site is designated in bold (amino acid position 363). The B2 glycosylation site is designated in bold (amino acid position 403). The linker sequence amino acids are designated in bold (amino acid positions 264 to 271).

FIG. 5 shows the nucleic acid sequence of the TST10092 construct (SEQ ID No. 5). The native proricin secretion signal is designated in bold (nucleotide positions −117 to −13). The A1 glycosylation site was mutated from Asn to Gln and is designated in bold (nucleotide positions 40 to 42). The A2 glycosylation site is designated in bold at nucleotide positions 718 to 720. The B1 glycosylation site is designated in bold at nucleotide positions 1087 to 1089. The B2 glycosylation site is designated in bold (nucleotide position 1207 to 1209). The linker sequence is designated in bold (nucleotide positions 790 to 813). The KEX-2 cleavage signal is designated in bold (nucleotide positions −13 to −1).

In the TST10147 construct (see FIGS. 3 and 6 (SEQ ID No. 3 and 6, respectively), there are two sequons (A2 and B1). In the yeast expression system, the A2 site in the TST10147 construct is not glycosylated, and thus the expressed protein is only glycosylated at one site (B1).

FIG. 3 shows the amino acid sequence of the TST10147 construct. The residual KEX2 cleavage amino acids (Glu-Ala-Glu-Ala) are designated in bold (amino acid positions 1 to 4). The A1 glycosylation site was mutated from Asn to Gln and is designated in bold (amino acid position 14). The A2 glycosylation site is designated in bold (amino acid position 240). The B1 glycosylation site is designated in bold (amino acid position 364). The B2 glycosylation site was mutated from Asn to Gln and is designated in bold (amino acid position 404). The linker sequence amino acids are designated in bold (amino acid positions 264 to 272).

FIG. 6 shows the nucleic acid sequence of the TST10147 construct (SEQ ID No. 6). The native proricin secretion signal is designated in bold (nucleotide positions −117 to −13). The A1 glycosylation site was mutated from Asn to Gln and is designated in bold (nucleotide positions 40 to 42). The A2 glycosylation site is designated in bold (nucleotide positions 718 to 720). The B1 glycosylation site is designated in bold (nucleotide positions 1090 to 1092). The B2 glycosylation site was mutated from Asn to Gln and is designated in bold (nucleotide position 1210 to 1212). The linker sequence is designated in bold (nucleotide positions 790 to 816). The KEX-2 cleavage signal is designated in bold (nucleotide positions −13 to −1).

### 1(a) Combinatorial Mutagenesis of Glycosylation Sites: Natural Gene Sequence and Codon Optimized Gene

In the natural ricin molecules there are four known sites of carbohydrate attachment: sequons A1, A2, B1 and B2. The relative positions of the sequons is shown in FIG. 7. Two cryptic sequons, designated B3 and B4, were also found in the amino acid sequence and mutations were made to determine their potential activity as well. It is now clear that the cryptic sites are not glycosylated in the recombinants. The DNA clones are referred to here as pPIC and the corresponding proteins are known as TST. A high degree of hyperglycosylation was observed in proteins produced from clones which used the α-mating factor secretion signal (TST10007 to TST10087). Alternatively, hyperglycosylation was virtually eliminated in clones that employed the ricin secretion signal (TST10088 to TST10092). The key amino acid at the linkage position is shown next to the clone name. Amino acids in parentheses indicate mutations at other (non-linkage) positions in the sequon. Glycosylation competent sequons (in yeast) are shown in blue. Positions that are not glycosylated are shown in yellow. See Table 1 and 2.

### 1(b) Glycosylation Pattern

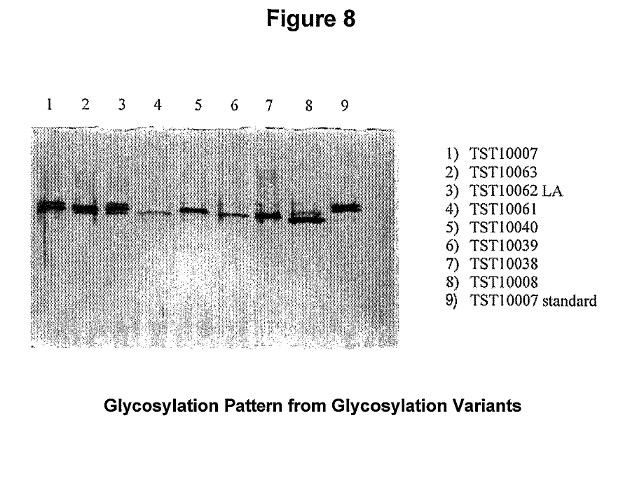

Glycosylation variants were characterized using Western blot analyses. FIG. 8 shows the glycosylation pattern of a subset of variants possessing different combinations of sequons. Five different species of protein are observed by Western Blot/PAGE. From the top of the gel to bottom these species are; i) hyperglycosylated material (appears as a smear in most lanes), ii) 3 glycosylation sites occupied (distinct band at top of triplet in TST10062), iii) 2 glycosylation sites occupied, iv) 1 glycosylation site occupied, v) no glycosylation (distinct band in TST10008). The two sequons on the A-chain are referred to as A1 and A2; while the two on the B-chain are referred to as B1 and B2. It is clear from the results that TST10007 (natural A-chain and B-chain sequences, all four sequons are available) is a protein with predominantly either 2 and 3 carbohydrate chains attached. The A1 sequon was only glycosylated 30% of the time and A2 was essentially never glycosylated. In shake flask fermentations both sequons on the B-chain are glycosylated. When the same variant is expressed in fermentor culture, however, there is evidence of heterogeneity in B-chain glycosylation.

FIG. 8 shows five different species of protein as observed by Western Blot/PAGE. From the top of the gel to bottom these species are: i) hyperglycosylated material (appears as a smear in most lanes), ii) 3 glycosylation sites occupied (distinct band a top of triplet in TST10062), iii) 2 glycosylation sites occupied, iv) 1 glycosylation site occupied, v) no glycosylation (distinct band in TST10008).

### 1(c) Efficacy of Glycoform 1 Against P388

In further studies, the A1 sequon was modified to prevent glycosylation of the A-chain, modification of A2 was unnecessary as it is never glycosylated in Pichia pastoris, in any event. The variant referred to as Glycoform 0 (TST10077) has been modified at the three competent positions (again A2 changes were unnecessary) to produce a protein without any carbohydrate attached. The variant referred to as Glycoform 1 (TST10086 which is essentially identical to TST10088—variants differing only in secretion signal) is solely glycosylated at the B1 position whereas Glycoform 2 (TST10087 which is essentially identical to TST10092—variants differing only in secretion signal) is glycosylated at both B1 and B2 positions.

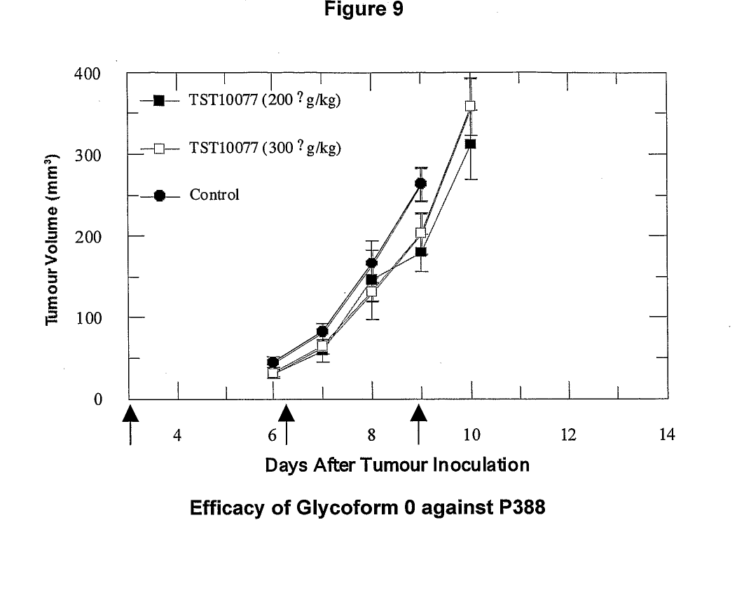

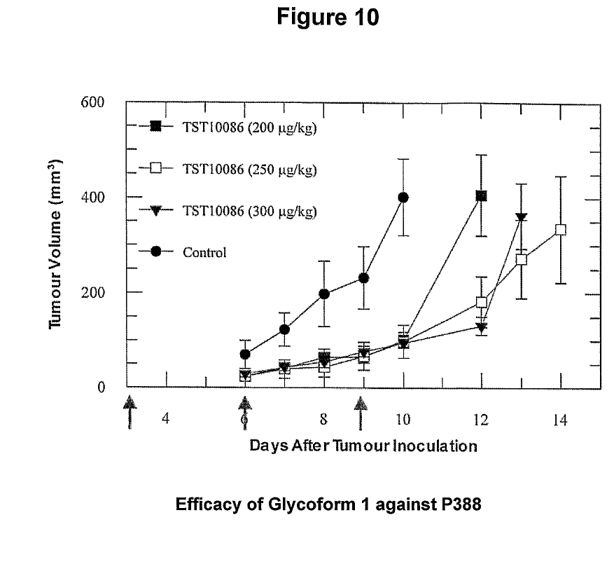

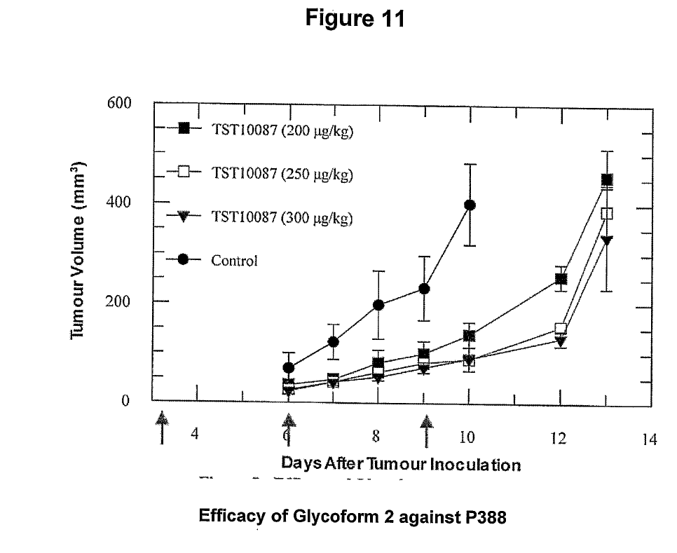

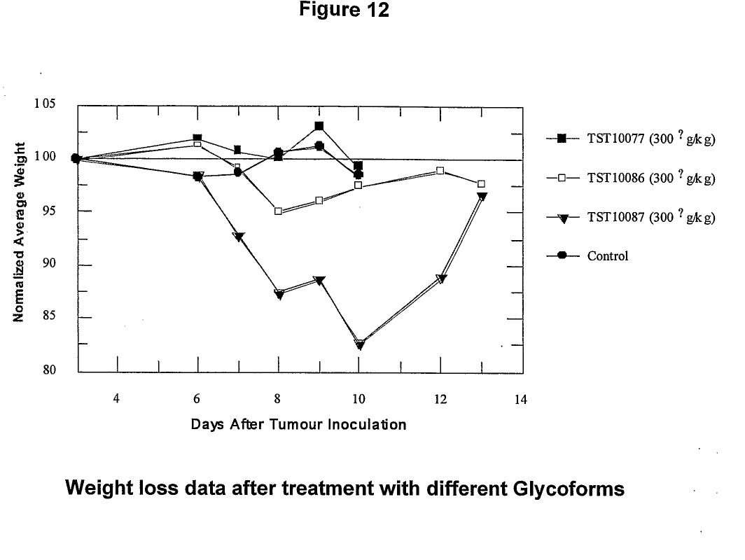

The activities of the three different glycoforms were investigated in the P388 animal model and results are shown in FIGS. 9, 10 and 11. FIG. 9 shows a P388 subcutaneous tumor model treated with TST10077. Treatments were made i.v. (n=4). FIG. 10 shows a P388 subcutaneous tumour model treated with TST10086 (protein identical to TST10088). Treatments were injected i.v. on days 3, 6 and 9 (n=4). FIG. 11 shows a P388 subcutaneous tumour model treated with TST10087 (protein identical to TST10092). Treatments were injected i.v. on days 3, 6 and 9 (n=4). FIG. 12 shows weight loss data in a P388 subcutaneous tumour model treated with TST10077, TST10086 and TST10087. Treatments were injected i.v. on days 3, 5 and 9 (n=4). Glycoform 1 and Glycoform 2 appear to have the same efficacy, but Glycoform 2 was found to be much more toxic than Glycoform 1 (i.e., higher weight loss—see FIG. 12). Therefore, it was determined that TST10088 (Glycoform 1) was a better molecule to take forward into preclinical development, because of the reduced toxicity.

The inventors therefore established that a minimum of one carbohydrate chain (i.e., at a single glycosylation site on the protein's B-chain) is essential to the activity of the prodrug. A protein completely devoid of carbohydrate has diminished activity (FIG. 9). Moreover, unglycosylated proteins were very difficult to express suggesting that they are very unstable. The inventors believe that non-glycosylated proteins become misdirected during uptake, whereas some carbohydrate is necessary to determine an appropriate route of uptake and localization to the endoplasmic reticulum. It is also interesting to note that a protein extensively glycosylated at all available sites has increased toxicity relative to the monoglycosylated species (FIG. 12).

### 1(d) Glycosylation Pattern and Secretion Signal

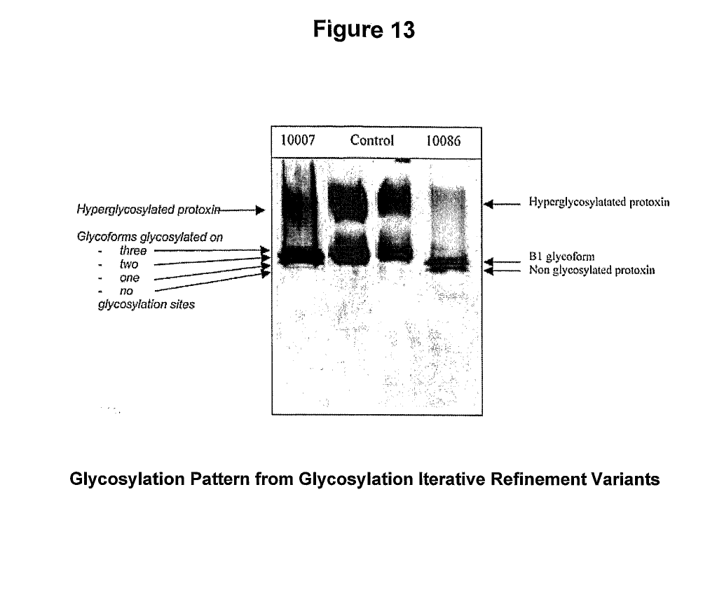

Yields of the finished product from fermentations of TST10007 were typically poor because of extensive hyperglycosylation (i.e., >70% of the secreted protein). Hyperglycosylated material was removed from downstream purification steps. In the case of TST10086, product yields were relatively improved as there was only one functioning glycosylation site and hyperglycosylation was reduced to roughly 10% of the secreted protein. FIG. 13 shows a silver stained SDS-PAGE gel comparing the fermentation end products of TST10007 and TST10086 (i.e., crude, unpurified products). Silver stained SDS-PAGE gels comparing the fermentation end products of TST10007 and TST10086 (i.e., crude, unpurified products). TST10007 is able to be glycosylated at three sites. TST10086 has only the B1 glycosylation site available. Samples each of 500 ng of TST10007 and TST10086 were analyzed. The control sample contains 500 ng of double and triple glycosylated protoxin and 500 ng of hyperglycosylated protoxin derived from previous fermentations.

TST10007 is able to be glycosylated at three sites. TST10086 has only the B1 glycosylation site available. Samples each of 500 ng of TST10007 and TST10086 were analyzed. The control sample contains 500 ng of double and triple glycosylated protoxin and 500 ng of hyperglycosylated protoxin derived from previous fermentations.

Gene constructs typically employed the α-mating factor secretion signal from Saccharomyces cerevisiae (TST 10007 through TST10087) to drive protein expression and secretion. Different secretion signals were eventually tested in an effort to improve expression levels. The best results were obtained using the natural ricin secretion signal. Moreover, the Applicant discovered that, in addition to improved overall protein yields, virtually all hyperglycosylation was eliminated (less than 5%) when genes were expressed using the ricin secretion signal (i.e., TST10088).

### 1(e) Signal Sequence and Cytotoxicity

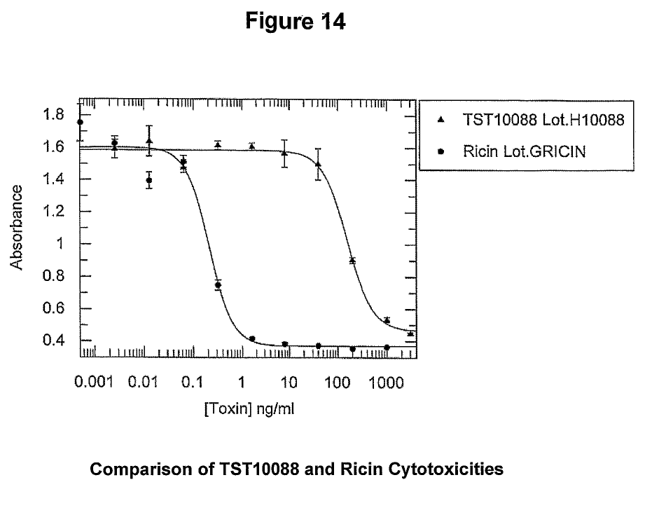

The purified proteins TST10086 and TST10088 are identical in all respects with the exception that the α-mating factor secretion signal was used to drive the production TST10086 and the ricin secretion signal was used to produce TST10088. Note that despite differences in the secretion signal, the molecules are processed the same and amino-termini of the two proteins is identical. The COS-1 cell cytotoxicity of TST10088 is indistinguishable from TST10086 and the molecules are interchangeable in animal studies. Cytotoxicity data for TST10088 is shown in FIG. 14 and Table 3 shows the lot-to-lot consistency of batches of research product.

### Example 2

### Combination Therapies

In essentially all approved anticancer therapies, drugs are used in combination. Since the inventors' compounds have a different mechanism of action from most conventional chemotherapeutics, they may potentiate the activity of conventional agents. TST10088 and TST10007 were tested in combination with various conventional chemotherapeutic agents to determine the extent to which they are able to potentiate the activity of other drugs.

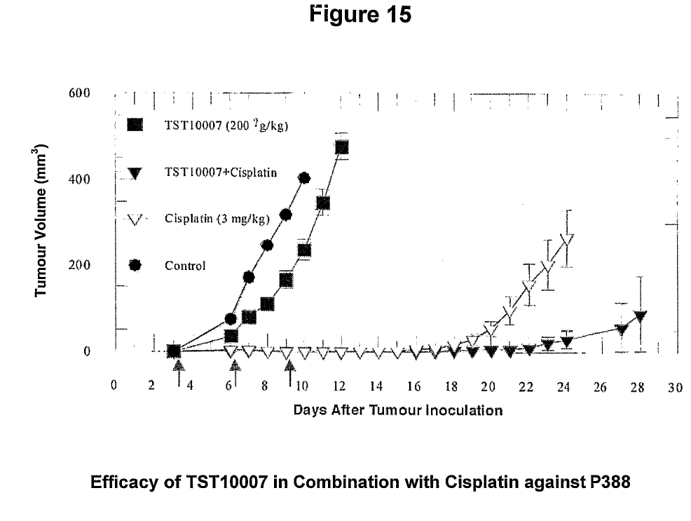

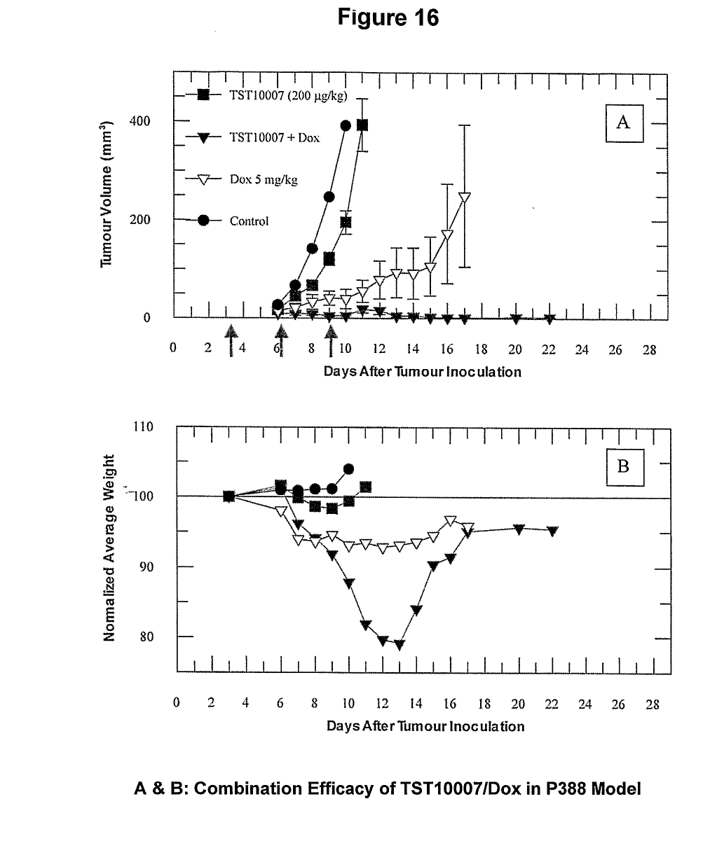

Cisplatin and doxorubicin were tested in combination with the mixed glycoform TST10007 (FIG. 15 and FIG. 16). FIG. 15 shows a P388 subcutaneous tumour model treated with TST10007 and the conventional drug cisplatin. Treatments were injected i.v. on days 3, 6 and 9 (n=4). FIG. 16A shows efficacy of TST10007 at 200 μg/kg (MTD=350 μg/kg) alone and in combination with doxorubicin, and FIG. 16B shows corresponding weight loss/toxicity of therapy. Animals were given 5 injections of drug or saline (controls) at 3 day intervals beginning on day three. The results showed that the effect of the combination treatment was greater than the sum of the individual monotherapies. However, the degree of supradditivity was not as great with cisplatin as that observed with doxorubicin.

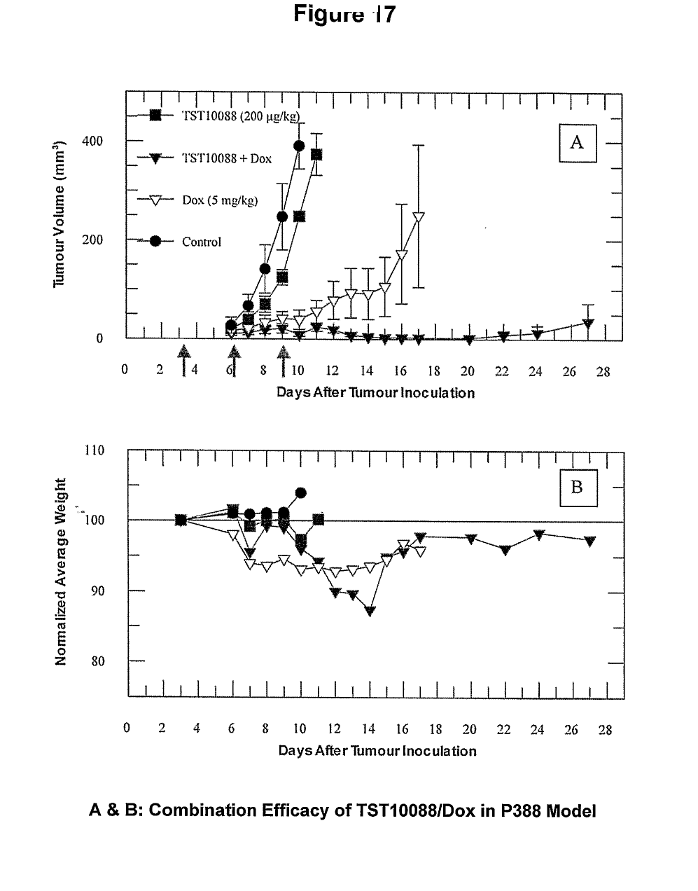

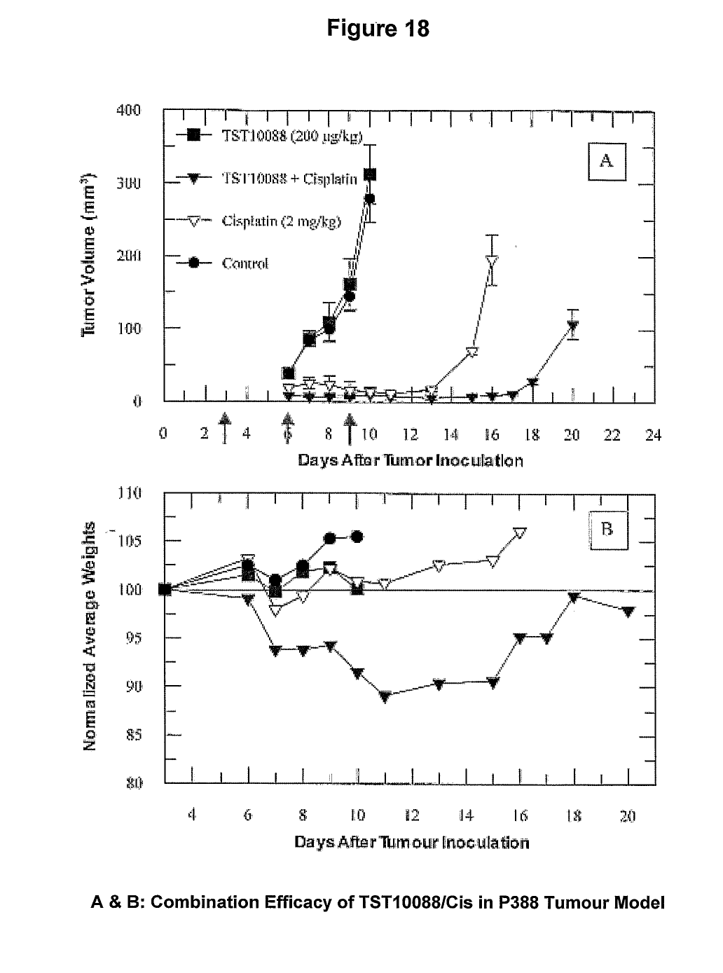

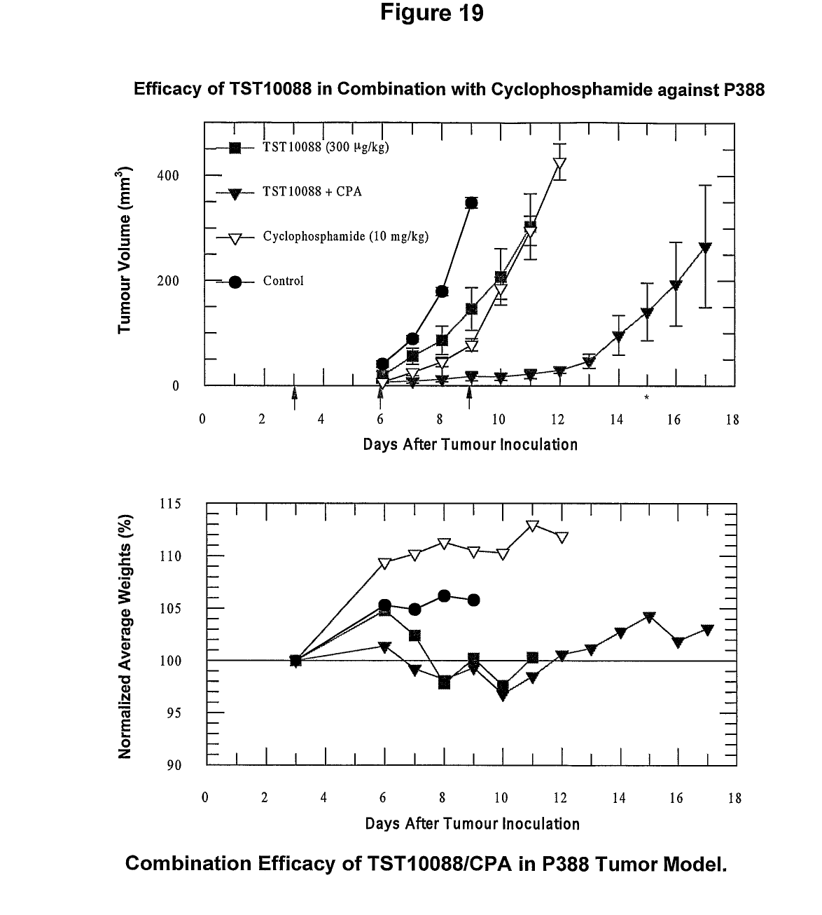

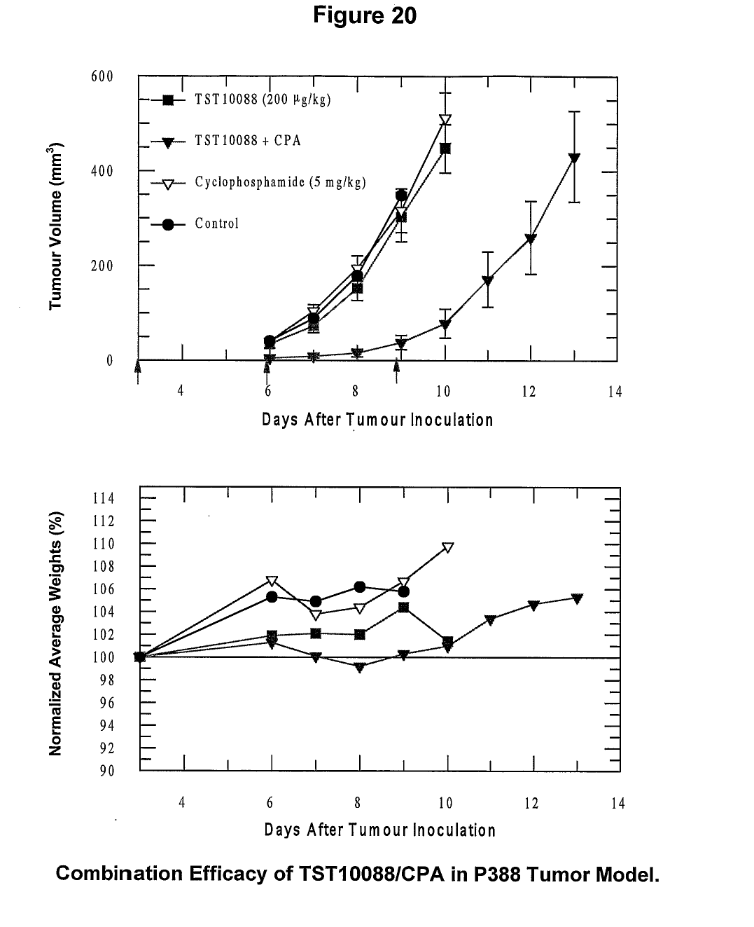

The studies outlined below showed a positive interaction between TST10088 and conventional agents: (i) doxorubicin (FIG. 17), (ii) cisplatin (FIG. 18), and (iii) cyclophosphamide (FIGS. 19 & 20). FIG. 17A shows the efficacy of TST10088 alone and in combination with doxorubicin, and FIG. 17B shows corresponding weight loss/toxicity of therapy. Animals were given 3 injections of drug or saline (controls) at 3 day intervals beginning on day three. FIG. 18A shows efficacy of TST10088 alone and in combination with cisplatin (i.p.), and FIG. 18B shows corresponding weight loss/toxicity of therapy. Treatments were given i.v. on days 3, 6 and 9 (n=4). FIG. 19 shows efficacy of TST10088 alone and in combination with cyclophosphamide (10 mg/kg). Treatments with TST10088 were injected i.v. and the conventional drug cyclophosphamide was injected i.p. Treatments (i.v./i.p.) were made on days 3, 6 and 9 (n=4). FIG. 20 shows efficacy of TST10088 alone and in combination with cyclophosphamide (5 mg/kg). Treatments with TST10088 were injected i.v. and the conventional drug cyclophosphamide was injected i.p. FIG. 20B shows corresponding weight loss. The treatments were injected on days 3, 6, and 9 (n=4).

The results, in particular the doxorubicin and cisplatin combinations, suggest that the molecules work synergistically (or more accurately, the response is superadditive). The effect of TST10088 in combination with cisplatin was once again more than additive. This finding was consistent with the results obtained with TST10007. The response to TST10088 combinations is consistent with previous observations of a strong positive interaction between TST334 and doxorubicin.

These combination studies further underscore the importance of glycosylation. Efficacy and weight loss data is shown for TST 10088 (single glycoform) and TST10007 (heterogeneous, multiple glycoforms) in FIGS. 17 and 16, respectively. Though TST10088 and TST10007 have similar efficacy at 200 μg/kg, TST10007 causes roughly twice the weight loss in animals with the combination. Thus, it was shown that TST10088 (Glycoform 1) had comparable efficacy to TST10007 in the P388 model, but reduced toxicity.

### Example 3

### Pharmacokinetic Analysis of TST10088+/−Doxorubicin

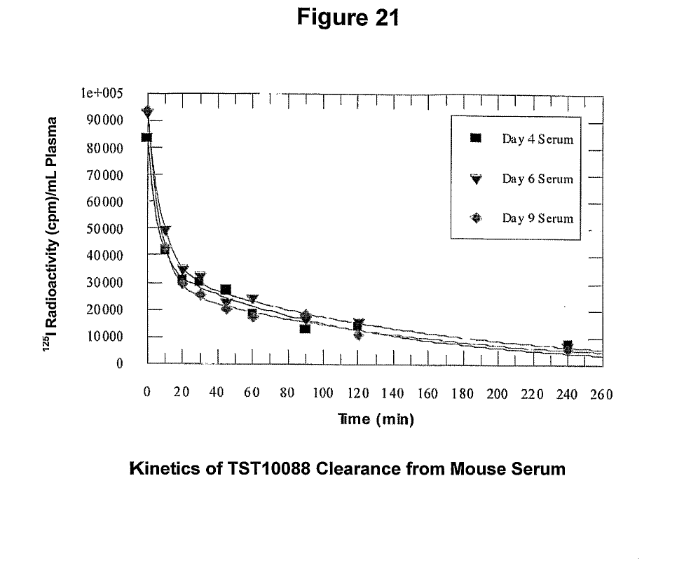

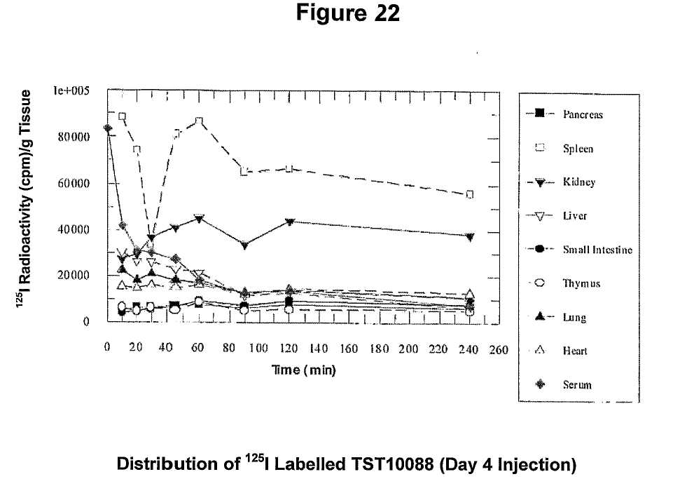

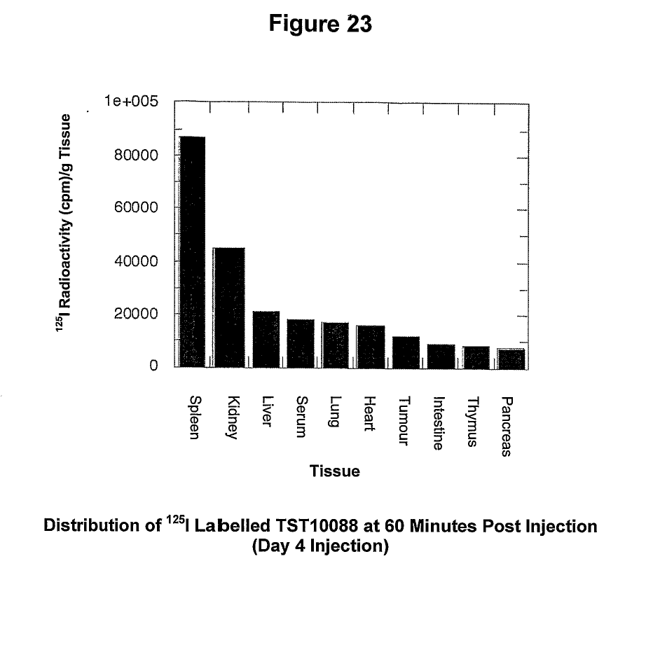

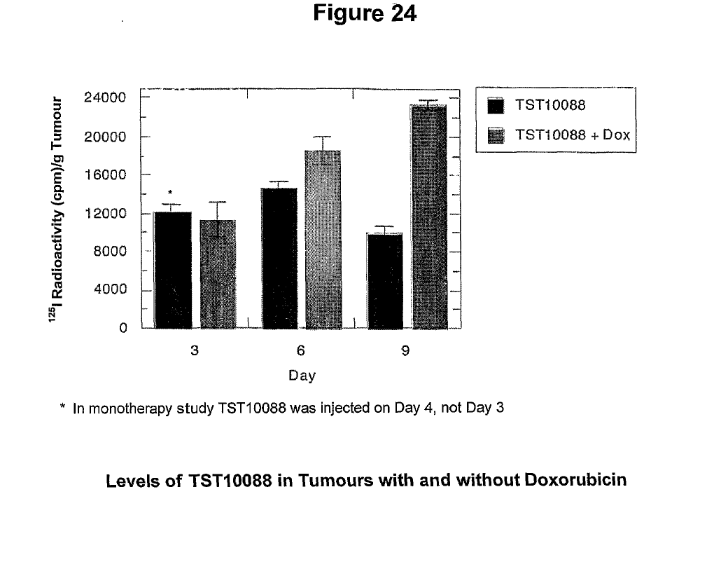

Pharmacokinetic studies have been performed with ¹²⁵I labeled TST10088 in female BDF1 mice. Illustrated in FIG. 21, the kinetics of TST10088 clearance is shown over three injections (also see Table 4). It is clear from the results the rate of clearance does not change during the period of the three injections. The distribution and clearance from tissues is shown in FIG. 22. Consistent with studies of the native ricin, the highest levels of label were found in the spleen. FIG. 23 shows the tissue levels 60 minutes post injection of TST10088. The results show that TST10088 is reaching the tumour. FIG. 24 shows that the amount of TST10088 that reached the tumour is relatively constant over three injections (days 4, 6 and 9). However, when TST10088 was injected in combination with doxorubicin, the amount of TST10088 in the tumour increases over time. This result may explain in part the greater than additive results observed when the two compounds were used together.

### Example 4

### Immune Response

Being foreign proteins, the inventors' prodrugs are capable of eliciting an immune response in humans. However, prior studies—in one case humans trials and the natural ricin and in another example humans and the related protein viscumin—suggest that there is a broad window of opportunity before a monotherapy is compromised by the immune response. Depending upon the treatment regimen this window could be as long as six weeks.

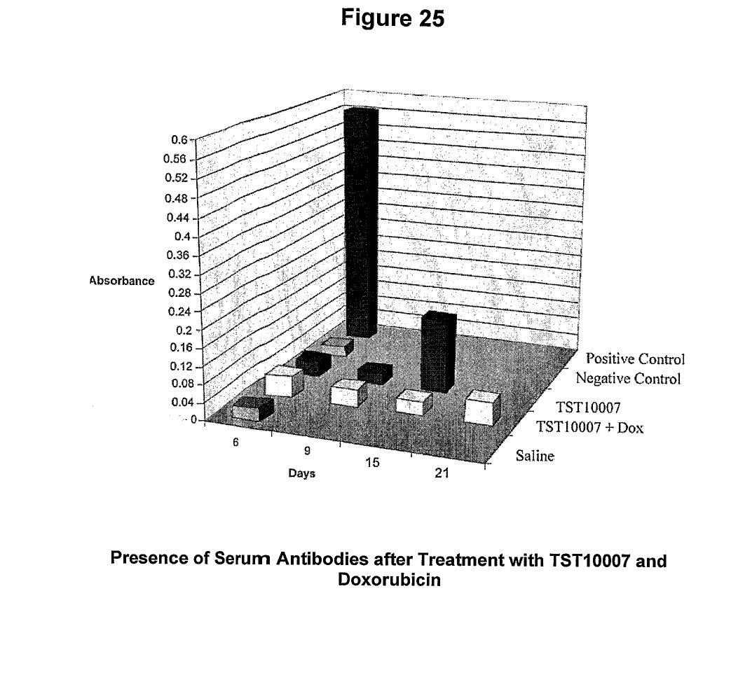

The inventors propose to take advantage of the immunosuppressive activity of combination agents such as doxorubicin and cisplatin to extend the treatment window beyond the six week horizon. To demonstrate the feasibility of this treatment approach, the inventors conducted studies measuring antibody titres in mice treated with prodrug in monotherapy and prodrug in combination. Results with TST10007 (see FIG. 25) show that in BDF1 mice an immune response was not seen until after the third treatment on day 9. Subsequent studies have shown that the immune response was seen as early as day 10 (data not shown). In combination with doxorubicin, however, the immune response was effectively suppressed during the course of treatment. These findings are consistent with the results reported by Fodstad (Godal, A., O. Fodstad, et al. (1983) Int J Cancer 32(4): 515-521) of ricin in combination with cyclophosphamide.

FIG. 25 shows a P388 subcutaneous tumour model treated with TST10007 and the conventional drug doxorubicin. Treatments were injected i.v. on days 3, 6, 9 for TST10007 and days 3, 6, 9 15 and 21 for TST10007 and doxorubicin (n=4). In the monotherapy and combination groups animals were sacrificed on the days indicated and anti-TST10088 antibodies determined.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

TABLE 1
Glycosylation Variants Part 1

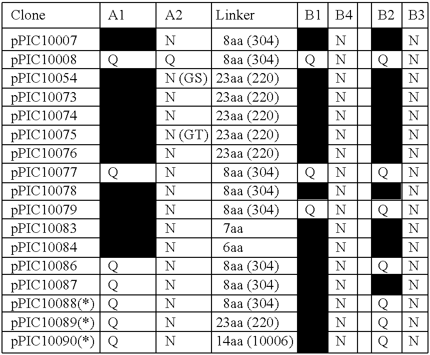

*These constructs use the proricin secretion signal

TABLE 2
Glycosylation Variants Part 2
Combinatorial Mutagenesis of Glycosylation Sites

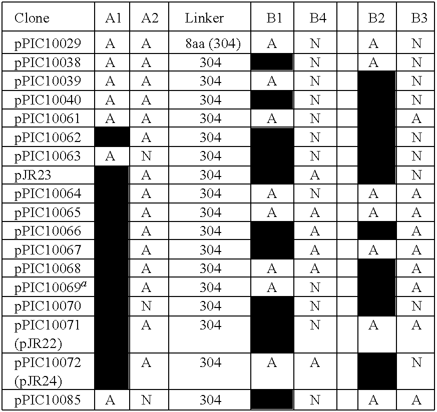

^{a}not cloned

TABLE 3
Lot-to-Lot Variability in the Activity of TST10088

| Toxin | IC50 (ng/mL) |
|---|---|
| Ricin Lot. GRICIN | 0.21 ± 0.05 |
| TST10088 Lot. H10088 | 157 ± 10.2 |
| Lot. G10088 | 122.9 ± 17.3 |
| Lot. F10088 | 68.1 ± 10 |
| Lot. E10088 | 113.1 ± 8.9 |
| Lot. D10088 | 116.4 ± 7.2 |
| Lot. C10088 | 113.7 ± 31 |
| Lot. A10088 | 105.3 ± 30.1 |

TABLE 4
Kinetics of TST10088 Clearance From Mouse Serum
Double Exponential Decay

| Variable | Day 4 | Day 6 | Day 9 |
|---|---|---|---|
| Initial 1 | 46978 | 57972 | 64645 |
| k1 (min⁻¹) | 0.17 | 0.12 | 0.14 |
| Initial 2 | 36696 | 35097 | 29210 |
| k2 (min⁻¹) | 0.0090 | 0.0070 | 0.0070 |

1: A recombinant protein comprising (a) an A chain of a ricin-like toxin, (b) a B chain of a ricin-like toxin and (c) a heterologous linker amino acid sequence linking the A and B chains, the linker sequence containing a cleavage recognition site for a disease-specific protease, wherein the A chain or the B chain has at least one glycosylation site.

2: The recombinant protein according to claim 1 wherein one or more glycosylation sites have been mutated and can not be glycosylated.

3: The recombinant protein according to claim 1, wherein the B chain has at least one glycosylation site.

4: The recombinant protein according to of claim 1, wherein only the B chain is glycosylated at B1.

5: The recombinant protein according to claim 1, wherein the recombinant protein has a ricin secretion signal sequence.

6: The recombinant protein according to claim 1, wherein the recombinant protein has the amino acid sequence shown in FIG. 1 (SEQ ID No. 1) or a fragment or analog thereof.

7: The recombinant protein according to claim 1, wherein the recombinant protein has the amino acid sequence shown in FIG. 2 (SEQ ID No. 2) or a fragment or analog thereof.

8: The recombinant protein according to claim 1, wherein the recombinant protein has the amino acid sequence shown in FIG. 3 (SEQ ID No. 3) or a fragment or analog thereof.

9: A purified and isolated nucleic acid molecule comprising (a) a nucleotide sequence encoding an A chain of a ricin-like toxin, (b) a nucleotide sequence encoding a B chain of a ricin-like toxin and (c) a nucleotide sequence encoding a heterologous linker amino acid sequence linking the A and B chain, the heterologous linker sequence containing a cleavable recognition site for a disease-specific protease, wherein the nucleotide sequence encoding the A chain or the nucleotide sequence encoding the B chain encodes an amino acid having at least one glycosylation site.

10: The nucleic acid molecule according to claim 9 wherein one or more glycosylation sites have been mutated and can not be glycosylated.

11: The nucleic acid molecule according to claim 9, wherein the nucleotide sequence of the B chain encodes an amino acid having at least one glycosylation site.

12: The nucleic acid molecule according to claim 9, wherein the nucleotide sequence of the B chain encodes an amino acid at B1 having a glycosylation site.

13: The nucleic acid molecule according to claim 9, wherein the nucleic acid molecule encodes a ricin secretion signal sequence.

14: The nucleic acid molecule according to claim 9 comprising:
(a) a nucleic acid sequence as shown in FIG. 4 (SEQ. ID. NO.:4), FIG. 5 (SEQ. ID. NO.:5) or FIG. 6 (SEQ. ID. NO.:6) wherein T can also be U;
(b) a nucleic acid sequence that is complementary to a nucleic acid sequence of (a);
(c) a nucleic acid sequence that has substantial sequence homology to a nucleic acid sequence of (a) or (b);
(d) a nucleic acid sequence that is an analog of a nucleic acid sequence of (a), (b) or (c); or
(e) a nucleic acid sequence that hybridizes to a nucleic acid sequence of (a), (b), (c) or (d) under stringent hybridization conditions.

15: The nucleic acid molecule according to claim 14, wherein the nucleic acid molecule has the nucleic acid sequence shown in FIG. 4 (SEQ ID No. 4).

16: The nucleic acid molecule according to claim 14, wherein the nucleic acid molecule has the nucleic acid sequence shown in FIG. 5 (SEQ ID No. 5).

17: The nucleic acid molecule according to claim 14, wherein the nucleic acid molecule has the nucleic acid sequence shown in FIG. 6 (SEQ ID No. 6).

18: A method of inhibiting or destroying cells affected by a disease, which cells are associated with a protease specific to the disease comprising the steps of:
(a) preparing a purified and isolated nucleic acid of claims 9;
(b) introducing the nucleic acid into a host cell and expressing the nucleic acid in the host cell to obtain a recombinant protein according to claim 1;
(c) suspending the protein in a pharmaceutically acceptable carrier, diluent or excipient, and
(d) contacting the cells with the recombinant protein.

19-28. (canceled)

29: A process for preparing a pharmaceutical composition for treating a mammal with cancer, fungal infection, viral infection or parasitic infection, comprising the steps of:
(a) preparing a purified and isolated nucleic acid according to claim 9, wherein the linker sequence contains a cleavage recognition site for a cancer, fungal or viral or parasitic protease;
(b) introducing the nucleic acid into a host cell and expressing the nucleic acid in the host cell to obtain a recombinant protein of claim 1;
(c) suspending the protein in a pharmaceutically acceptable carrier, diluent or excipient.

30: A process for preparing a pharmaceutical composition for treating a mammal with cancer, comprising the steps of:
(a) preparing a purified and isolated nucleic acid according to claim 9, wherein the linker sequence contains a cleavage recognition site for a cancer protease;
(b) introducing the nucleic acid into a host cell and expressing the nucleic acid in the host cell to obtain a recombinant protein of claim 1;
(c) suspending the protein in a pharmaceutically acceptable carrier, diluent or excipient.

31: The process according to claim 29, wherein the pharmaceutical composition further comprise at least one additional anticancer therapy.

32: A process according to claim 31, wherein the additional anticancer therapy is one or more of the following: doxorubicin, cisplatin, cyclophosphamide etoposide, paclitaxel, taxotere, carboplatin, oxaliplatin, 5-fluorouracil, irinotecan, topotecan, vincristine, gemcitabine, epirubicin, capecitabine, and temozolomide.

33: A pharmaceutical composition for treating cancer or a fungal, viral, or parasitic infection in an animal comprising the recombinant protein of claim 1 and a pharmaceutically acceptable carrier, diluent or excipient.

34: A pharmaceutical composition for treating cancer or a fungal, viral or parasitic infection in any animal comprising the nucleic acid molecule of claim 9 and a pharmaceutically acceptable carrier, diluent or excipient.

35: A pharmaceutical composition for treating cancer according to claim 33, further comprising at least one additional anticancer therapy.

36: A pharmaceutical composition according to claim 35, wherein the additional anticancer therapy is one or more of the following: doxorubicin, cisplatin, cyclophosphamide etoposide, paclitaxel, taxotere, carboplatin, oxaliplatin, 5-fluorouracil, irinotecan, topotecan, vincristine, gemcitabine, epirubicin, capecitabine, and temozolomide.

37: A method of inhibiting or destroying cells affected by a disease, which cells are associated with a protease specific to the disease comprising administering a recombinant protein according to claim 1 to a cell or animal in need thereof.

38: The method according to claim 37, wherein the disease is cancer.

39: The method according to claim 38, further comprising using at least one additional anticancer therapy.

40: The method according to claim 39, wherein the additional anticancer therapy is one or more of the following: doxorubicin, cisplatin, cyclophosphamide etoposide, paclitaxel, taxotere, carboplatin, oxaliplatin, 5-fluorouracil, irinotecan, topotecan, vincristine, gemcitabine, epirubicin, capecitabine, and temozolomide.

41: The method according to claim 37 wherein the disease is a viral, fungal or parasitic infection.

42: A method of inhibiting or destroying cells affected by a disease, which cells are associated with a protease specific to the disease, comprising administering a nucleic acid molecule according to claim 9 to a cell or animal in need thereof.

43: The method according to claim 42, wherein the disease is cancer.

44: The method according to claim 43, further comprising using at least one additional anticancer therapy.

45: The method according to claim 44, wherein the additional anticancer therapy is one or more of the following: doxorubicin, cisplatin, cyclophosphamide etoposide, paclitaxel, taxotere, carboplatin, oxaliplatin, 5-fluorouracil, irinotecan, topotecan, vincristine, gemcitabine, epirubicin, capecitabine, and temozolomide.

46: The method according to claim 42 wherein the disease is a viral, fungal or parasitic infection.


SEQUENCE LISTING (6 sequences)

SEQ ID NO: 1 - protein, 530 aa, Artificial Sequence
  TST10088 Protein Sequence
EAEAIFPKQYPIIQFTTAGATVQSYTNFIRAVRGRLTTGADVRHEIPVLPNRVGLPINQRFILVELSNHAELSVTLALDVTNAYVVGYRAGNSAYFFHPDNQEDAEAITHLFTDVQNRYTFAFGGNYDRLEQLAGNLRENIELGNGPLEEAISALYYYSTGGTQLPTLARSFIICIQMISEAARFQYIEGEMRTRIRYNRRSAPDPSVITLENSWGRLSTAIQESNQGAFASPIQLQRRNGSKFSVYDVSILIPIIALMVYRCSPQGIAGQCMDPEPIVRIVGRNGLCVDVRDGRFHNGNAIQLWPCKSNTDANQLWTLKRDNTIRSNGKCLTTYGYSPGVYVMIYDCNTAATDATRWQIWDNGTIINPRSSLVLAATSGNSGTTLTVQTNIYAVSQGWLPTQNTQPFVTTIVGLYGLCLQANSGQVWIEDCSSEKAEQQWALYADGSIRPQQNRDNCLTSDSNIRETVVKILSCGPASSGQRWMFKNDGTILNLYSGLVLDVRASDPSLKQIILYPLHGDPNQIWLPLF

SEQ ID NO: 2 - protein, 530 aa, Artificial Sequence
  TST10092 Protein Sequence
EAEAIFPKQYPIIQFTTAGATVQSYTNFIRAVRGRLTTGADVRHEIPVLPNRVGLPINQRFILVELSNHAELSVTLALDVTNAYVVGYRAGNSAYFFHPDNQEDAEAITHLFTDVQNRYTFAFGGNYDRLEQLAGNLRENIELGNGPLEEAISALYYYSTGGTQLPTLARSFIICIQMISEAARFQYIEGEMRTRIRYNRRSAPDPSVITLENSWGRLSTAIQESNQGAFASPIQLQRRNGSKFSVYDVSILIPIIALMVYRCSPQGIAGQCMDPEPIVRIVGRNGLCVDVRDGRFHNGNAIQLWPCKSNTDANQLWTLKRDNTIRSNGKCLTTYGYSPGVYVMIYDCNTAATDATRWQIWDNGTIINPRSSLVLAATSGNSGTTLTVQTNIYAVSQGWLPTNNTQPFVTTIVGLYGLCLQANSGQVWIEDCSSEKAEQQWALYADGSIRPQQNRDNCLTSDSNIRETVVKILSCGPASSGQRWMFKNDGTILNLYSGLVLDVRASDPSLKQIILYPLHGDPNQIWLPLF

SEQ ID NO: 3 - protein, 531 aa, Artificial Sequence
  TST10147 Protein Sequence
EAEAIFPKQYPIIQFTTAGATVQSYTNFIRAVRGRLTTGADVRHEIPVLPNRVGLPINQRFILVELSNHAELSVTLALDVTNAYVVGYRAGNSAYFFHPDNQEDAEAITHLFTDVQNRYTFAFGGNYDRLEQLAGNLRENIELGNGPLEEAISALYYYSTGGTQLPTLARSFIICIQMISEAARFQYIEGEMRTRIRYNRRSAPDPSVITLENSWGRLSTAIQESNQGAFASPIQLQRRNGSKFSVYDVSILIPIIALMVYRCGSPQGIAGQCMDPEPIVRIVGRNGLCVDVRDGRFHNGNAIQLWPCKSNTDANQLWTLKRDNTIRSNGKCLTTYGYSPGVYVMIYDCNTAATDATRWQIWDNGTIINPRSSLVLAATSGNSGTTLTVQTNIYAVSQGWLPTQNTQPFVTTIVGLYGLCLQANSGQVWIEDCSSEKAEQQWALYADGSIRPQQNRDNCLTSDSNIRETVVKILSCGPASSGQRWMFKNDGTILNLYSGLVLDVRASDPSLKQIILYPLHGDPNQIWLPLF

SEQ ID NO: 4 - DNA, 1707 bp, Artificial Sequence
  TST10088 DNA Insert Sequence
atgaaaccgggaggaaatactattgtaatatgggtgtatgcagtggcaacatggctttgttttggatccacctcagggtggtctttcacattagaggataacaacctcgagaaaagagaggctgaagctatattccccaaacaatacccaattatacagtttaccacagcgggtgccactgtgcaaagctacacaaactttatcagagctgttcgcggtcgtttaacaactggagctgatgtgagacatgaaataccagtgttgccaaacagagttggtttgcctataaaccaacggtttattttagttgaactctcaaatcatgcagagctttctgttacattagcgctggatgtcaccaatgcatatgtggtcggctaccgtgctggaaatagcgcatatttctttcatcctgacaatcaggaagatgcagaagcaatcactcatcttttcactgatgttcaaaatcgatatacattcgcctttggtggtaattatgatagacttgaacaacttgctggtaatctgagagaaaatatcgagttgggaaatggtccactagaggaggctatctcagcgctttattattacagtactggtggcactcagcttccaactctggctcgttcctttataatttgcatccaaatgatttcagaagcagcaagattccaatatattgagggagaaatgcgcacgagaattaggtacaaccggagatctgcaccagatcctagcgtaattacacttgagaatagttgggggagactttccactgcaattcaagagtctaaccaaggagcctttgctagtccaattcaactgcagagacgtaatggttccaaattcagtgtgtacgatgtgagtatattaatccctatcatagctctcatggtgtatagatgctctccgcaaggaattgcagggcagtgtatggatcctgagcccatagtgcgtatcgtaggtcgaaatggtctatgtgttgatgttagggatggaagattccacaacggaaacgcaatacagttgtggccatgcaagtctaatacagatgcaaatcagctctggactttgaaaagagacaatactattcgatctaatggaaagtgtttaactacttacgggtacagtccgggagtctatgtgatgatctatgattgcaatactgctgcaactgatgccacccgctggcaaatatgggataatggaaccatcataaatcccagatctagtctagttttagcagcgacatcagggaacagtggtaccacacttacagtgcaaaccaacatttatgccgttagtcaaggttggcttcctactcagaatacacaaccttttgtgacaaccattgttgggctatatggtctgtgcttgcaagcaaatagtggacaagtatggatagaggactgtagcagtgaaaaggctgaacaacagtgggctctttatgcagatggttcaatacgtcctcagcaaaaccgagataattgccttacaagtgattctaatatacgggaaacagttgtcaagatcctctcttgtggccctgcatcctctggccaacgatggatgttcaagaatgatggaaccattttaaatttgtatagtgggttggtgttagatgtgagggcatcagatccgagccttaaacaaatcattctttaccctctccatggtgacccaaaccaaatatggttaccattattt

SEQ ID NO: 5 - DNA, 1707 bp, Artificial Sequence
  TST10092 DNA Insert Sequence
atgaaaccgggaggaaatactattgtaatatgggtgtatgcagtggcaacatggctttgttttggatccacctcagggtggtctttcacattagaggataacaacctcgagaaaagagaggctgaagctatattccccaaacaatacccaattatacagtttaccacagcgggtgccactgtgcaaagctacacaaactttatcagagctgttcgcggtcgtttaacaactggagctgatgtgagacatgaaataccagtgttgccaaacagagttggtttgcctataaaccaacggtttattttagttgaactctcaaatcatgcagagctttctgttacattagcgctggatgtcaccaatgcatatgtggtcggctaccgtgctggaaatagcgcatatttctttcatcctgacaatcaggaagatgcagaagcaatcactcatcttttcactgatgttcaaaatcgatatacattcgcctttggtggtaattatgatagacttgaacaacttgctggtaatctgagagaaaatatcgagttgggaaatggtccactagaggaggctatctcagcgctttattattacagtactggtggcactcagcttccaactctggctcgttcctttataatttgcatccaaatgatttcagaagcagcaagattccaatatattgagggagaaatgcgcacgagaattaggtacaaccggagatctgcaccagatcctagcgtaattacacttgagaatagttgggggagactttccactgcaattcaagagtctaaccaaggagcctttgctagtccaattcaactgcagagacgtaatggttccaaattcagtgtgtacgatgtgagtatattaatccctatcatagctctcatggtgtatagatgctctccgcaaggaattgcagggcagtgtatggatcctgagcccatagtgcgtatcgtaggtcgaaatggtctatgtgttgatgttagggatggaagattccacaacggaaacgcaatacagttgtggccatgcaagtctaatacagatgcaaatcagctctggactttgaaaagagacaatactattcgatctaatggaaagtgtttaactacttacgggtacagtccgggagtctatgtgatgatctatgattgcaatactgctgcaactgatgccacccgctggcaaatatgggataatggaaccatcataaatcccagatctagtctagttttagcagcgacatcagggaacagtggtaccacacttacagtgcaaaccaacatttatgccgttagtcaaggttggcttcctactaataatacacaaccttttgtgacaaccattgttgggctatatggtctgtgcttgcaagcaaatagtggacaagtatggatagaggactgtagcagtgaaaaggctgaacaacagtgggctctttatgcagatggttcaatacgtcctcagcaaaaccgagataattgccttacaagtgattctaatatacgggaaacagttgtcaagatcctctcttgtggccctgcatcctctggccaacgatggatgttcaagaatgatggaaccattttaaatttgtatagtgggttggtgttagatgtgagggcatcagatccgagccttaaacaaatcattctttaccctctccatggtgacccaaaccaaatatggttaccattattt

SEQ ID NO: 6 - DNA, 1710 bp, Artificial Sequence
  TST10147 DNA Insert Sequence
atgaaaccgggaggaaatactattgtaatatgggtgtatgcagtggcaacatggctttgttttggatccacctcagggtggtctttcacattagaggataacaacctcgagaaaagagaggctgaagctatattccccaaacaatacccaattatacagtttaccacagcgggtgccactgtgcaaagctacacaaactttatcagagctgttcgcggtcgtttaacaactggagctgatgtgagacatgaaataccagtgttgccaaacagagttggtttgcctataaaccaacggtttattttagttgaactctcaaatcatgcagagctttctgttacattagcgctggatgtcaccaatgcatatgtggtcggctaccgtgctggaaatagcgcatatttctttcatcctgacaatcaggaagatgcagaagcaatcactcatcttttcactgatgttcaaaatcgatatacattcgcctttggtggtaattatgatagacttgaacaacttgctggtaatctgagagaaaatatcgagttgggaaatggtccactagaggaggctatctcagcgctttattattacagtactggtggcactcagcttccaactctggctcgttcctttataatttgcatccaaatgatttcagaagcagcaagattccaatatattgagggagaaatgcgcacgagaattaggtacaaccggagatctgcaccagatcctagcgtaattacacttgagaatagttgggggagactttccactgcaattcaagagtctaaccaaggagcctttgctagtccaattcaactgcagagacgtaatggttccaaattcagtgtgtacgatgtgagtatattaatccctatcatagctctcatggtgtatagatgcggttctccgcaaggaattgcagggcagtgtatggatcctgagcccatagtgcgtatcgtaggtcgaaatggtctatgtgttgatgttagggatggaagattccacaacggaaacgcaatacagttgtggccatgcaagtctaatacagatgcaaatcagctctggactttgaaaagagacaatactattcgatctaatggaaagtgtttaactacttacgggtacagtccgggagtctatgtgatgatctatgattgcaatactgctgcaactgatgccacccgctggcaaatatgggataatggaaccatcataaatcccagatctagtctagttttagcagcgacatcagggaacagtggtaccacacttacagtgcaaaccaacatttatgccgttagtcaaggttggcttcctactcagaatacacaaccttttgtgacaaccattgttgggctatatggtctgtgcttgcaagcaaatagtggacaagtatggatagaggactgtagcagtgaaaaggctgaacaacagtgggctctttatgcagatggttcaatacgtcctcagcaaaaccgagataattgccttacaagtgattctaatatacgggaaacagttgtcaagatcctctcttgtggccctgcatcctctggccaacgatggatgttcaagaatgatggaaccattttaaatttgtatagtgggttggtgttagatgtgagggcatcagatccgagccttaaacaaatcattctttaccctctccatggtgacccaaaccaaatatggttaccattattt